(19) United States
(12) Patent Application Publication
(10) Pub. No.: US 2022/0106627 A1
(43) Pub. Date: Apr. 7, 2022

(54) METHODS OF DIAGNOSING TUBERCULOSIS AND DIFFERENTIATING BETWEEN ACTIVE AND LATENT TUBERCULOSIS

(71) Applicant: Cepheid, Sunnyvale, CA (US)

(72) Inventor: Malin Nygren, Solna (SE)

(73) Assignee: Cepheid, Sunnyvale, CA (US)

(21) Appl. No.: 17/494,427

(22) Filed: Oct. 5, 2021

Related U.S. Application Data
(60) Provisional application No. 63/104,187, filed on Oct. 22, 2020.
(60) Provisional application No. 63/088,077, filed on Oct. 6, 2020.

(51) Int. Cl. C12Q 1/689; C12Q 1/6851

(52) U.S. Cl. CPC C12Q 1/689; C12Q 1/6851; C12Q 2600/112; G01N 2333/9723; G01N 2333/522; G01N 2333/55; G01N 2333/811; C12Q 2600/158

(57) ABSTRACT

Compositions and methods for detecting Mycobacterium tuberculosis (MTB) infection in a patient suspected of being infected with Mycobacterium tuberculosis and for distinguishing between active and latent tuberculosis infection are provided. The methods may also be used to monitor progression of MTB infection or to monitor treatment of MTB infected patients. Changes in the expression level of genes are used to aid in the diagnosis, prognosis and treatment of tuberculosis.

### FIELD OF THE DISCLOSURE

Compositions and methods for diagnosing tuberculosis are provided. In particular, the disclosure relates to markers and panels of markers useful to detect patients that are infected with Mycobacterium tuberculosis (MTB) and also distinguish between latent tuberculosis infection (LTB or LTBI) and active tuberculosis (ATB) in a single assay.

### BACKGROUND

Tuberculosis (TB) is a worldwide public health issue, with 9 million new infections and 1.5 million deaths in 2018 (Global Tuberculosis Programme, World Health Organization. Global tuberculosis report. Geneva, Switzerland: World Health Organization; 2019). Despite advances in diagnosis and treatment, there is still a large burden of disease and there are an estimated 1.7 billion people with latent tuberculosis. It is important to identify patients with LTBI as approximately 5-10% will progress to ATB in their lifetime if untreated. TB is difficult to accurately diagnose; traditional methods such as tuberculin skin testing and interferon gamma release assays (IGRAs) are unable to distinguish between latent TB infection (LTBI) and active TB (ATB), and have lower sensitivity in HIV-positive patients. The XPERT® MTB/RIF assay (CEPHEID®, Sunnyvale, Calif.) assay is a PCR test and has significantly improved diagnostic power for ATB. This assay is optimized to use induced sputum, which can be difficult to obtain from adults after symptomatic improvement or from pediatric patients at any time. Current methods could thus potentially be complemented by a blood-based diagnostic and treatment-response test that can be used to detect both latent and active infections and to distinguish between ATB and LTBI, preferably in a single assay.

Better methods of monitoring responses to treatment and for predicting which patients are at risk for progression to ATB from LTBI are also needed. Furthermore, obtaining results in a short amount of time, allowing for different sample types and utilization of fewer reagents is beneficial.

### SUMMARY

Compositions and methods for identifying in individuals the presence or absence of tuberculosis (TB) and further determining if those individuals that are infected with TB have ATB or LTBI are disclosed. In particular, markers and panels of markers useful in detecting and differentiating between ATB and LTBI and between MTB and not infected are disclosed. In some instances, methods are provided for the use of biomarkers for diagnosis of tuberculosis status in a single assay. In particular, the inventors have discovered a single set of biomarkers that can be measured and that a first subset of the single set can be used to detect the presence of a tuberculosis infection and a second subset of the single set can be used to distinguish ATB from LTBI in infected patients. These biomarkers can be used alone or in combination with one or more additional biomarkers or relevant clinical parameters in prognosis, diagnosis, assessing risk for progression, or monitoring treatment of tuberculosis. Based on the diagnosis patients may be provided Tuberculosis Preventive Treatment (TPT) and optionally a full-course treatment for ATB. In some embodiments, the biomarkers for analysis are selected from IFN-γ, MIG, IP10, IL2, FoxP3, PLAU, SLPI, VEGFA, DUSP3, GBP5, GBP1P1, ANKRD22, SERPING1, PTGS2, and IL10. The mRNA levels of the selected biomarkers are measured, for example, by quantitative RT-PCR, and the results can be analyzed in a first analysis to determine if the individual is or has been infected with Mycobacterium tuberculosis (MTB) and in a second analysis to determine if infected individuals have LTBI or ATB. In some embodiments, the levels of mRNA for the markers are normalized to one or more endogenous controls. Internal controls such as housekeeping genes may be used. In some embodiments, the endogenous control is selected from ABL mRNA, TBP mRNA and CD3E mRNA. In some embodiments, an endogenous control is selected that is expected to be expressed at similar levels in samples from subjects with and without ATB or LTBI. In some embodiments, the sample is a blood sample. In some embodiments the sample may be normalized to levels of T-cell makers such as CD3E, CD4 or CD8 or normalized to a fixed number of PBMCs.

In some embodiments one or more additional biomarker selected from TNFA, TGFA, IL2RA, IL8, IL12B, CISH, FLT1, LINC01093, KLF2, PRDX1, CCL7 is added to the set of biomarkers that are measured and analyzed.

In one aspect, the disclosure includes a method for diagnosing and treating a patient suspected of being infected with MTB, the method comprising: a) obtaining a biological sample from the patient; measuring the expression of a set of genes in the patient, wherein the set of genes shows changes in expression in response to an active tuberculosis infection or the presence of latent tuberculosis. The change in expression may be overexpression or under expression and may vary from gene to gene. In some embodiments the genes are selected from the following biomarkers: IFN-γ, MIG, IP10, IL2, FoxP3, PLAU, SLPI, VEGFA, DUSP3, GBP5, GBP1P1, ANKRD22, SERPING1, PTGS2, and IL10 The biomarkers to be analyzed may include a first signature that is used to diagnose the patient as being TB infected or not infected and a second signature that is used to diagnose TB infected patients as having an active or latent infection. The biomarkers in the first and second signatures may be entirely different or there may be an overlap of one or more biomarkers. For biomarkers that are common to both signatures they may be weighted differently in the first and second signatures. The patient may be diagnosed as either infected or not infected with MTB and for patients that are infected a diagnosis of ATB or LTBI may be made by analysis of the data from the same test by analyzing the levels of expression of one or more of the biomarkers in conjunction with respective reference value ranges for a control, wherein increased levels of expression of the set of genes that are overexpressed in patients who have tuberculosis compared to the reference value ranges for the control optionally in combination with decreased levels of expression of the set of genes that are under expressed in patients who have tuberculosis compared to the reference value ranges for the control subject indicate that the patient has tuberculosis. Depending on the diagnosis a patient may be triaged toward TPT or a full-course of treatment for ATB. For example, after a diagnosis is made an effective amount of at least one antibiotic may be administered to the patient if the patient is diagnosed with tuberculosis. The selection of antibiotic and the duration of treatment may be selected based on the diagnosis.

In some embodiments at least 2, at least 3, at least 4, at least 5, at least 6, at least 7, at least 8, at least 9 or at least 10 biomarkers are analyzed and each signature comprises at least 2, at least 3, at least 4, at least 5, at least 6, at least 7, or at least 8 biomarkers. In some assays pairs of markers can be detected in the same channel with the same dye. This would preferably be used in the event the 2 markers are interchangeable because they are differentially expressed based on variation in individual immune response.

In some embodiments, the expression of VEGFA, PLAU, DUSP3, GBP5, GBP1P1, IL2, MIG, SLPI, and IFN-γ are measured, and the markers used to diagnose MTB infection comprise 2 or more of VEGFA, GBP1P1, MIG, IL2 and IFN-γ and the markers used to diagnose ATB or LTBI comprises 2 or more of VEGFA, IL2, GBP1P1, GBP5, DUSP3, PLAU, and SLPI. In another embodiment, the expression of VEGFA, PLAU, DUSP3, GBP5, GBP1P1, IL2, MIG, SLPI, and IFN-γ are measured and the markers used to diagnose MTB infection comprises GBP1P1, MIG, IL2 and IFN-γ and the markers used to diagnose ATB or LTBI comprises VEGFA, GBP1P1, GBP5, DUSP3, PLAU, and SLPI.

In some embodiments the markers used to diagnose MTB infection are IFN-γ, MIG, IL2, FOXP3, and GBP1P1, with optionally the addition of one or more markers selected from DUSP3, PLAU and SLPI and the markers used to diagnose ATB or LTBI are GBP5, SLPI, PLAU, IL2 with the optional addition of DUSP3. The second signature can include VEGFA, PLAU and IL2. In another embodiment the first signature includes IL2, MIG, FOXP3 and IFN-γ and the second signature includes VEGFA, PLAU, GBP5, FOXP3 and IL2.

In some embodiments IL2, PLAU and IFN-γ are measured and the first signature includes IL2 and IFN-γ and the second signature includes PLAU and IL2. In another embodiment the first signature includes IL2, MIG and IFN-γ and the second signature includes VEGFA, PLAU and IL2. In another embodiment the first signature includes IL2, MIG, FOXP3 and IFN-γ and the second signature includes VEGFA, PLAU, GBP5, FOXP3 and IL2.

In some embodiments the patient is (i) suspected of being infected with MTB, (ii) suspected of having ATB, (iii) at risk of having LTBI (for example HIV co-infected, household contacts of patient with ATB), (iv) being actively treated for ATB and being tested to monitor treatment response or (v) being treated with TPT and being tested to monitor treatment response.

In some embodiments a blood sample is stimulated by exposure to one or more antigens that are specific for MTB prior to measuring the expression levels of the biomarkers. This may be done in one or more tubes where one or more antigens are present on the inner surface of the tube. Multiple antigens may be present in a tube. A matrix carrier may be used as a substrate for attaching the antigen to the surface of the tube. In some embodiments the methods comprise stimulating a sample from the patient in a single tube with a mixture of antigens that allow for discrimination between ATB and LTBI and discrimination of patients infected with MTB from healthy subjects. The present disclosure also relates to a combination of MTB antigens that may be used to stimulate a blood sample prior to analysis for expression of the selected biomarkers. The blood is incubated with the antigens for at least 0.1 hour (e.g., about 3 hours) or for a longer period such as overnight prior to measuring the expression levels of the biomarkers. The antigens may include CFP-10, ESAT-6, TB7.7, Mb3645c, Rv3615c and Ala-DH or epitopes of these proteins. For examples of peptides that comprise epitopes that may be used see, for example, U.S. Pat. Nos. 8,697,091 and 9,005,902. The antigens may be recombinant proteins, synthetic peptides, or fragments of proteins or peptides and may be used individually or in combinations.

Both short (e.g., greater than 0 hours to less than 8 hours) and long (e.g., 8-24 hours) incubation of the blood with antigens can be used in the methods disclosed herein. The inventors have found that in some instances, the first signature which determines MTB infected vs. not infected may perform slightly better at long incubation, while the second signature which determines ATB vs. LTBI may perform better with the short incubation time. Therefore, depending on the incubation time, the disclosed methods may be optimized to use different assay protocols such as variable cycling conditions (e.g., temperature, flow volumes and rates, and incubation times), target genes (e.g., different combinations of targets in the assay, different weighting of the targets in the assay, or both), among others.

In some embodiments, the method comprises detecting an exogenous control. In some embodiments, the exogenous control is a sample processing control. In some embodiments, the exogenous control comprises an RNA sequence that is not expected to be present in the sample. In some embodiments, the exogenous control is an RNA control. In some embodiments, the RNA control is packaged in a bacteriophage protective coat (e.g., ARMORED® RNA). In some embodiments, the method comprises contacting nucleic acids from the sample with a control primer pair for detecting an exogenous control.

In some embodiments, the method comprises PCR. In some embodiments, the method comprises quantitative PCR. In some embodiments the method comprises RT-PCR, wherein RNA is reverse transcribed to create cDNA and cDNA is amplified by PCR. In some embodiments, the RT-PCR reaction takes less than 2 hours from an initial denaturation step through a final extension step. In some embodiments, the reaction takes less than 2 hours, less than 1 hour, less than 45 minutes, less than 40 minutes, less than 35 minutes, or less than 30 minutes from initial denaturation through the last extension.

In some embodiments, the method comprises contacting nucleic acids from the sample with a primer pair for detecting each of the biomarkers. In some embodiments, the primer pair comprises a first primer and a second primer, wherein the first primer comprises a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of each biomarker, and wherein the second primer comprises a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of each biomarker.

In some embodiments the sample is whole blood, sputum, peripheral blood mononuclear cells (PBMCs), monocytes, or macrophages. The sample may be collected in a tube containing heparin and lithium.

In some embodiments, the method comprises forming an amplicon from each primer pair when the target of the primer pair is present. In some embodiments, each primer pair produces an amplicon that is 50 to 500 nucleotides long, 50 to 400 nucleotides long, 50 to 300 nucleotides long, 50 to 200 nucleotides long, or 50 to 150 nucleotides long.

In some embodiments, the method comprises contacting the amplicons with at least one probe. In some embodiments, the probe comprises a sequence that is at least 85%, at least 90%, at least 95%, or 100% identical or complementary to at least 13, at least 14, at least 15, at least 16, at least 17, at least 18, at least 19, at least 20, at least 21, at least 22, at least 23, at least 24, or at least 25 contiguous nucleotides of the biomarker. In some embodiments the method comprises contacting the amplicons with a probe for each biomarker to be analyzed.

In some embodiments, each probe comprises a detectable label. In some embodiments, each probe comprises a fluorescent dye and a quencher molecule. In some embodiments, the probes comprise detectable labels that are detectably different. In some embodiments, the probes comprise detectable labels that are not detectably different. In some embodiments, each probe consists of 13 to 30 nucleotides.

In some embodiments, the method comprises forming an exogenous control amplicon. In some embodiments, the method comprises contacting the exogenous control amplicon with a control probe capable of selectively hybridizing with the exogenous control amplicon.

In some embodiments, compositions for preforming an RT-PCR reaction are provided.

In some embodiments, the composition further comprises a probe for detecting an exogenous control. In some embodiments, each probe comprises a detectable label. In some embodiments, each probe comprises a fluorescent dye and a quencher molecule. In some embodiments, each probe consists of 15 to 30 nucleotides.

In some embodiments, the composition is a lyophilized composition. In some embodiments, the composition is in solution. In some embodiments, the composition comprises nucleic acids from a sample from a subject being tested for the presence or absence of tuberculosis.

In some embodiments, kits are provided. In some embodiments, a kit comprises a composition described herein. In some embodiments, the kit further comprises an exogenous control. In some embodiments, the exogenous control is an RNA control. In some embodiments, the RNA control is packaged in a bacteriophage protective coat (e.g., ARMORED® RNA). In some embodiments, the kit comprises dNTPs and/or a thermostable polymerase. In some embodiments, the kit comprises a reverse transcriptase. In some embodiments the kit contains primers and probes for detecting an endogenous control RNA. In some embodiments the kit comprises a tube containing one or more MTB antigens. The antigens may be peptide epitopes, peptide analogues, natural or synthetic peptides and may be recombinant.

In some embodiments, one or more oligonucleotides are provided. In some embodiments, the oligonucleotide comprises at least one modified nucleotide. In some embodiments, the oligonucleotide comprises a detectable label. In some embodiments, the oligonucleotide comprises a fluorescent dye and a quencher molecule. In some embodiments, the oligonucleotide is a fluorescence resonance energy transfer (FRET) probe.

### BRIEF DESCRIPTION OF THE FIGURES

FIG. 1A shows a graph of the variable accuracy of each marker in a set of 15 markers analyzed using random forest modelling in MTB infected vs. not infected ranked from highest to lowest accuracy.

FIG. 1B shows a graph of the variable accuracy of each marker in a set of 15 markers analyzed using random forest modelling in ADB infected vs. LTBI infected ranked from highest to lowest accuracy.

### DETAILED DESCRIPTION

### Definitions

To facilitate an understanding of the present disclosure, a number of terms and phrases are defined below:

As used herein, the terms “detect”, “detecting” or “detection” may describe either the general act of discovering or discerning or the specific observation of a detectably labeled composition.

As used herein, the term “detectably different” refers to a set of labels (such as dyes) that can be detected and distinguished simultaneously.

As used herein, the terms “patient” and “subject” are used interchangeably to refer to a human. In some embodiments, the methods described herein may be used on samples from non-human animals.

As used herein, the terms “oligonucleotide,” “polynucleotide,” “nucleic acid molecule,” and the like, refer to nucleic acid-containing molecules, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “oligonucleotide,” refers to a single-stranded polynucleotide having fewer than 500 nucleotides. In some embodiments, an oligonucleotide is 8 to 200, 8 to 100, 12 to 200, 12 to 100, 12 to 75, or 12 to 50 nucleotides long. Oligonucleotides may be referred to by their length, for example, a 24 residue oligonucleotide may be referred to as a “24-mer.”

As used herein, the term “complementary” to a target RNA (or target region thereof), and the percentage of “complementarity” of the probe sequence to that of the target RNA sequence is the percentage “identity” to the sequence of target RNA or to the reverse complement of the sequence of the target RNA. In determining the degree of “complementarity” between probes used in the compositions described herein (or regions thereof) and a target RNA, such as those disclosed herein, the degree of “complementarity” is expressed as the percentage identity between the sequence of the probe (or region thereof) and sequence of the target RNA or the reverse complement of the sequence of the target RNA that best aligns therewith. The percentage is calculated by counting the number of aligned bases that are identical as between the 2 sequences, dividing by the total number of contiguous nucleotides in the probe, and multiplying by 100. When the term “complementary” is used, the subject oligonucleotide is at least 90% complementary to the target molecule, unless indicated otherwise. In some embodiments, the subject oligonucleotide is at least 91%, at least 92%, at least 93%, at least 94%, at least 95%, at least 96%, at least 97%, at least 98%, at least 99%, or 100% complementary to the target molecule.

A “primer” or “probe” as used herein, refers to an oligonucleotide that comprises a region that is complementary to a sequence of at least 8 contiguous nucleotides of a target nucleic acid molecule, such as DNA (e.g., a target gene) or an mRNA (or a DNA reverse-transcribed from an mRNA). In some embodiments, a primer or probe comprises a region that is complementary to a sequence of at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of a target molecule. When a primer or probe comprises a region that is “complementary to at least x contiguous nucleotides of a target molecule,” the primer or probe is at least 95% complementary to at least x contiguous nucleotides of the target molecule. In some embodiments, the primer or probe is at least 96%, at least 97%, at least 98%, at least 99%, or 100% complementary to the target molecule.

The term “nucleic acid amplification,” encompasses any means by which at least a part of at least one target nucleic acid is reproduced, typically in a template-dependent manner, including without limitation, a broad range of techniques for amplifying nucleic acid sequences, either linearly or exponentially. Exemplary means for performing an amplifying step include polymerase chain reaction (PCR), ligase chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase polymerase amplification and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), digital amplification, and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 Feb.; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf Dis. 2:18-(2002); Lage et al., Genome Res. 2003 Feb.; 13(2):294-307, and Landegren et al., Science 241:1077-80 (1988), Demidov, V., Expert Rev Mol Diagn. 2002 Nov.; 2(6):542-8., Cook et al., J Microbiol Methods. 2003 May; 53(2):165-74, Schweitzer et al., Curr Opin Biotechnol. 2001 Feb.; 12(1):21-7, U.S. Pat. Nos. 5,830,711, 6,027,889, 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

Unless otherwise indicated, the term “hybridize” is used herein refer to “specific hybridization” which is the binding, duplexing, or hybridizing of a nucleic acid molecule preferentially to a particular nucleotide sequence, in some embodiments, under stringent conditions. The term “stringent conditions” refers to conditions under which a probe will hybridize preferentially to its target sequence, and to a lesser extent to, or not at all to, other sequences. A “stringent hybridization” and “stringent hybridization wash conditions” in the context of nucleic acid hybridization (e.g., as in array, Southern, or Northern hybridization) are sequence-dependent and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions for filter hybridizations are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Dependency of hybridization stringency on buffer composition, temperature, and probe length are well known to those of skill in the art (see, e.g., Sambrook and Russell (2001) Molecular Cloning: A Laboratory Manual (3rd ed.) Vol. 1-3, Cold Spring Harbor Laboratory, Cold Spring Harbor Press, NY).

An “antigen,” as used herein, includes any substance that evokes an immune response either alone or after forming a complex with one or more other molecules. An epitope is a portion of an antigen molecule to which an antibody attaches or which is capable of eliciting an immune response.

A “sample,” or “biological sample” as used herein, includes various samples of tissue, cells, or fluid isolated from a subject, including but not limited to, for example, whole blood, buffy coat, plasma, serum, immune cells (e.g., monocytes or macrophages), and sputa. In some embodiments, the sample comprises a buffer, such as an anticoagulant, and/or a preservative. In some embodiments whole blood is mixed with heparin in a lithium heparin blood collection tube. The sample can be from any bodily fluid, tissue or cells that contain the expressed biomarker. A biological sample can be obtained from a subject by conventional techniques. For example, blood can be obtained by venipuncture or a finger-prick capillary, and solid tissue samples can be obtained by surgical techniques according to methods well known in the art. In some aspects the blood sample is placed into a tube that is specifically designed for the assay.

An “endogenous control,” as used herein refers to a moiety that is naturally present in the sample to be used for detection. In some embodiments, an endogenous control is a “sample adequacy control” (SAC), which may be used to determine whether there was sufficient sample used in the assay, or whether the sample comprised sufficient biological material, such as cells. In some embodiments, an endogenous control is an RNA (such as an mRNA, tRNA, ribosomal RNA, etc.), such as a human RNA. Nonlimiting exemplary endogenous controls include CD3E, TBP, CD4, CD8B, B2M, ABL mRNA, GUSB mRNA, GAPDH mRNA, TUBB mRNA, and UPK1a mRNA. In some embodiments, an endogenous control, such as a SAC, is selected that can be detected in the same manner as the target RNA is detected and, in some embodiments, simultaneously with the target RNA. Controls may be used both for relative quantitation, for example, normalizing gene expression levels of a marker and to establish a Ct cut-off value for specimen stability.

An “exogenous control,” as used herein, refers to a moiety that is added to a sample or to an assay, such as a “sample processing control” (SPC). In some embodiments, an exogenous control is included with the assay reagents. An exogenous control is typically selected that is not expected to be present in the sample to be used for detection, or is present at very low levels in the sample such that the amount of the moiety naturally present in the sample is either undetectable or is detectable at a much lower level than the amount added to the sample as an exogenous control. In some embodiments, an exogenous control comprises a nucleotide sequence that is not expected to be present in the sample type used for detection of the target RNA. In some embodiments, an exogenous control comprises a nucleotide sequence that is not known to be present in the species from whom the sample is taken. In some embodiments, an exogenous control comprises a nucleotide sequence from a different species than the subject from whom the sample was taken. In some embodiments, an exogenous control comprises a nucleotide sequence that is not known to be present in any species. In some embodiments, an exogenous control is selected that can be detected in the same manner as the target RNA is detected and, in some embodiments, simultaneously with the target RNA. In some embodiments, the exogenous control is an RNA. In some such embodiments, the exogenous control is an ARMORED® RNA, which comprises RNA packaged in a bacteriophage protective coat. See, e.g., WalkerPeach et al., Clin. Chem. 45:12: 2079-2085 (1999).

In the sequences herein, “U” and “T” are used interchangeably, such that both letters indicate a uracil or thymine at that position. One skilled in the art will understand from the context and/or intended use whether a uracil or thymine is intended and/or should be used at that position in the sequence. For example, one skilled in the art would understand that native RNA molecules typically include uracil, while native DNA molecules typically include thymine. Thus, where an RNA sequence includes “T”, one skilled in the art would understand that that position in the native RNA is likely a uracil.

In the present disclosure, “a sequence selected from” encompasses both “one sequence selected from” and “one or more sequences selected from.” Thus, when “a sequence selected from” is used, it is to be understood that one, or more than one, of the listed sequences may be chosen.

In the present disclosure, the phrase “level of expression” refers to expression of either mRNA or protein whose abundance is measured quantitatively.

The phrase “differentially expressed” refers to differences in the quantity and/or the frequency of a biomarker present in a sample taken from patients having, for example, tuberculosis as compared to a control subject or non-infected subject. For example, a biomarker can be a polynucleotide which is present at an elevated level or at a decreased level in samples of patients with MTB, ATB or LTBI compared to samples of control subjects. Alternatively, a biomarker can be a polynucleotide which is detected at a higher frequency or at a lower frequency in samples of patients with tuberculosis compared to samples of control subjects. A biomarker can be differentially present in terms of quantity, frequency or both.

A polynucleotide is differentially expressed between two samples if the amount of the polynucleotide in one sample is statistically significantly different from the amount of the polynucleotide in the other sample. For example, a polynucleotide is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polynucleotide is differentially expressed in two sets of samples if the frequency of detecting the polynucleotide in samples of patients infected with MTB is statistically significantly higher or lower than in the control samples or if the frequency of detection in samples with ATB is statistically significantly higher or lower than in the samples with LTBI. For example, a polynucleotide is differentially expressed in two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

A “biomarker” in the context of the present disclosure refers to a biological compound, such as a polynucleotide or polypeptide which is differentially expressed in a sample taken from patients having tuberculosis as compared to a comparable sample taken from control subjects (e.g., a person with a negative diagnosis, normal or healthy subject, or non-infected subject) or differentially expressed in a sample from a patient having ATB as compared to a sample from a patient having LTBI. The biomarker can be a nucleic acid, a fragment of a nucleic acid, a polynucleotide, or an oligonucleotide that can be detected and/or quantified. Tuberculosis biomarkers include polynucleotides comprising nucleotide sequences from genes or RNA transcripts of genes, including but not limited to, IFN-γ, MIG, IP10, IL2, FoxP3, PLAU, SLPI, VEGFA, DUSP3, GBP5, GBP1P1, ANKRD22, SERPING1, PTGS2, and IL10, and their expression products. The biomarkers may be selected from interferon gamma, monokine induced by gamma (also CXCL9 or C-X-C motif chemokine ligand 9), interferon gamma-induced protein 10 (also CXCL10 or C-X-C motif chemokine ligand 10), interleukin 2, forkhead box P3, plasminogen activator urokinase, synaptotagmin-like protein 1, vascular endothelial growth factor A, dual specificity phosphatase 3, guanylate binding protein 5, guanylate binding protein 1 pseudogene 1, ankyrin repeat domain 22, serpin family G member 1, prostaglandin-endoperoxide synthase 2, and interleukin 10.

An example of the nucleotide sequence of IFN-γ is published in the NCBI database under the accession number AC007458. An example of the nucleotide sequence of MIG (monokine induced by gamma or CXCL9) is published in the NCBI database under the accession number AEMK02000067. An example of the nucleotide sequence of IP10 (interferon γ-induced protein or CXCL10) is published in the NCBI database under the accession number AC112719. An example of the nucleotide sequence of IL2 (interleukin 2) is published in the NCBI database under the accession number AC022489. An example of the nucleotide sequence of FoxP3 (forkhead box P3) is published in the NCBI database under the accession number AC232271. An example of the nucleotide sequence of PLAU (plasminogen activator, urokinase) is published in the NCBI database under the accession number A21571. An example of the nucleotide sequence of SLPI (secretory leukocyte peptidase inhibitor) is published in the NCBI database under the accession number AL035660. An example of the nucleotide sequence of VEGFA (vascular endothelial growth factor A) is published in the NCBI database under the accession number AF092126. An example of the nucleotide sequence of DUSP3 (dual specificity phosphatase 3) is published in the NCBI database under the accession number AC003098. An example of the nucleotide sequence of GBP5 (guanylate binding protein 5) is published in the NCBI database under the accession number AC099063. An example of the nucleotide sequence of GBP1P1 (guanylate binding protein 1 pseudogene 1) is published in the NCBI database under the accession number AL691464. An example of the nucleotide sequence of ANKRD22 (ankyrin repeat domain 22) is published in the NCBI database under the accession number AL157394. An example of the nucleotide sequence of SERPING1 (serpin family G member 1) is published in the NCBI database under the accession number AF435921. An example of the nucleotide sequence of PTGS2 (prostaglandin-endoperoxide synthase 2) is published in the NCBI database under the accession number AF044206. An example of the nucleotide sequence of IL10 (interleukin 10) is published in the NCBI database under the accession number AB098711.

In general, interferon-gamma release-assays (IGRAs) are whole blood tests that can aid in diagnosing MTB infection. IGRAs measure a person's immune reactivity to M. tuberculosis after stimulation with MTB-specific antigens, such as early secretory antigen target-6 (ESAT-6) and culture filtrate protein 10 (CFP10) or other strong targets of T cells in MTB infection. These tests are based on the principle that T-cells of infected individuals produce IFN-γ and other proteins associated with a strong proinflammatory-like response when they re-encounter M. tuberculosis antigens. White blood cells from most persons that have been infected with M. tuberculosis will have a cellular immune response, characterized by the release of interferon-gamma (IFN-γ) when mixed with antigens (substances that can produce an immune response) derived from M. tuberculosis and absent from most non-tuberculous mycobacteria (NTM) or by Mycobacterium bovis Bacille Calmette-Guérin (BCG) To conduct the tests, fresh blood samples are mixed with antigens and controls. Commercially available tests include the QuantiFERON®-TB Gold Plus (QFT-Plus) and the T-SPOT® test. They may be performed on whole blood or peripheral blood mononuclear cells (PBMCs). The antigen or antigens used for stimulation may vary, commonly used antigens include, alanine dehydrogenase (Ala-DH), ESAT-6, CFP-10 and TB7.7 (and peptides derived from these antigens). Additional antigens have also been disclosed, see for example, U.S. Pat. No. 10,295,538. The antigens may be provided as recombinant proteins, immunologically active fragments (peptides, mimotope peptides or analogues thereof) and mixtures of synthetic peptides that may be derived from naturally occurring antigens. The antigens stimulate release of cytokines from both CD4+ and CD8+T cells. Recent studies suggest that a strong CD8+ T-cell response can be detected in ATB patients as well as patients coinfected with HIV so inclusion of antigens for the stimulation of CD8+ T-cells may improve sensitivity of detection of both latent and active TB. The tests typically measure the resulting levels of IFN-γ and other immune stimulation markers to determine if the patient is infected with MTB and can typically be run in less than 24 hours. Other MTB antigens that may be used include PstS1, HSPX and antigen 85B. A multi-biomarker approach, such as described herein, may allow for improved diagnosis in patients with compromises immune function (e.g. impaired T-cell function and reduced CD4 cell count), such as patients with HIV.

In some embodiments the antigens may include a peptide analogue of a naturally occurring peptide. Analogue peptides may comprise one or more modifications, that may be natural post-translational modifications or artificial modifications. The modification may provide a chemical moiety (typically by substitution of a hydrogen, e.g. of a C—H bond), such as an amino, acetyl, hydroxy or halogen (e.g. fluorine) group or carbohydrate group. Typically, the modification is present on the N or C terminus. The analogue may comprise one or more non-natural amino acids, for example amino acids with a side chain different from natural amino acids. The non-natural amino acid may be an L-amino acid. The analogue typically has a shape, size, flexibility or electronic configuration which is substantially similar to the original peptide. It is typically a derivative of the original peptide.

Although diagnostic sensitivity of commercially available IGRAs is higher than skin tests, their real-life clinical use demands higher sensitivity to enable rapid exclusion of active tuberculosis and reliably diagnose latent tuberculosis in those at highest risk of progression to tuberculosis and who are at risk of false-negative IGRA results, i.e. people who are immunosuppressed by virtue of HIV-infection, concomitant chronic illness (e.g. end-stage renal failure, diabetes, immune-mediated inflammatory diseases) medication (e.g. corticosteroids, anti-TNF-alpha agents) or young age (children under 5-years and especially under 2-years of age). One approach is to increase diagnostic sensitivity by incorporating additional antigens that are strong targets of T cell responses in MTB-infected persons but not in BCG-vaccinated persons. Additional markers that may be released in response to these antigens include other cytokines and regulatory factors implicated in the pathogenesis and control of MTB infection including, for example, TNF-α, IL-2R, IL-4, IL-10, MIG, IP-10, and I-TAC. The mRNA expression levels of these markers can be correlated with the protein levels. Variation in patient immune response may result in failure of the classical T-cell response markers to identify patient status, and may be associated with worse patient outcome, so having additional responsive markers is desirable.

### Identifying Tuberculosis Infections

The present inventors have developed a combination assay for detecting individuals that are infected with tuberculosis and distinguishing between active tuberculosis infections and latent tuberculosis infections. In some embodiments, the assay comprises measuring the expression of a set of biomarkers and analyzing the set of markers to generate a first signature to diagnose the presence or absence of a tuberculosis infection and a second signature that differentiates between ATB and LTB. If the first signature is negative or inconclusive but the second signature is positive for ATB or LTBI, the second signature alone may be used for selecting treatment.

Candidate marker genes were identified from whole genome expression analysis using RNA-seq studies and analysis of previous studies and the candidate markers were assayed in samples of know MTB status to identify combinations of markers that can be used to diagnose MTB status reliably.

The biomarker can be a nucleic acid, a fragment of a nucleic acid, a polynucleotide, or an oligonucleotide that can be detected and/or quantified. Biomarkers that can be used in the practice of the disclosure include polynucleotides comprising nucleotide sequences from genes or RNA transcripts of genes, including but not limited to, IFN-γ, MIG, IP10, IL2, FoxP3, PLAU, SLPI, VEGFA, DUSP3, GBP5, GBP1P1, ANKRD22, SERPING1, PTGS2, and IL10, and their expression products. Differential expression of these biomarkers is associated with tuberculosis and therefore expression profiles of these biomarkers are useful for diagnosing MTB infection and distinguishing active tuberculosis from latent tuberculosis. The markers may be used in different combinations of 2 or more and different combinations may be used for diagnosing MTB infection and differentiating between ATB and LTBI. By way of example, IFN-γ, MIG, IL2 and VEGFA may be analyzed to differentiate between MTB infected and not-infected, and PLAU, SLP1, VEGFA and GBP5 may be analyzed to differentiate between ATB and LTBI. In this example, there are 7 biomarkers analyzed: 4 are used in the first analysis, 4 are used in the second analysis, and one biomarker, VEGFA, is included in both analyses.

Accordingly, in one aspect, the disclosure provides a method for diagnosing MTB status in a subject, comprising measuring the level of a plurality of biomarkers in a biological sample derived from a subject suspected of being infected with MTB, having ATB or LTBI, and analyzing the levels of the biomarkers and comparing with respective reference value ranges for the biomarkers, wherein differential expression of one or more biomarkers in the biological sample compared to one or more biomarkers in a control sample indicates that the subject has tuberculosis. Differential expression may be measured by comparing the Ct of the biomarker to the Ct of a control or reference marker to obtain a ΔCt value. When analyzing the levels of biomarkers in a biological sample, the reference value ranges used for comparison can represent the levels of one or more biomarkers found in one or more samples of one or more subjects without active tuberculosis (e.g., healthy subject, non-infected subject, or subject with latent tuberculosis). Alternatively, the reference value ranges can represent the levels of one or more biomarkers found in one or more samples of one or more subjects with active tuberculosis. In certain embodiments, the levels of the biomarkers in a biological sample from a subject are compared to reference values for subjects with latent or active tuberculosis.

In certain embodiments, a panel of biomarkers is used for diagnosis of tuberculosis. Biomarker panels of any size can be used in the practice of the disclosure. Biomarker panels for diagnosing tuberculosis typically comprise at least 3 biomarkers and up to 30 biomarkers, including any number of biomarkers in between, such as 3, 4, 5, 6, 7, 8, 9, 10, 11, 12, 13, 14, 15, 16, 17, 18, 19, 20, 21, 22, 23, 24, 25, 26, 27, 28, 29, or 30 biomarkers. In certain embodiments, the disclosure includes a biomarker panel comprising at least 2, at least 3, or at least 4, or at least 5, or at least 6, or at least 7, or at least 8, or at least 9, or at least 10, or at least 11 or more biomarkers. Although smaller biomarker panels are usually more economical, larger biomarker panels (i.e., greater than 30 biomarkers) have the advantage of providing more detailed information and can also be used in the practice of the disclosure.

In some embodiments the expression levels of each of a first panel of biomarkers is analyzed to diagnose the patient as having TB and the expression levels of each of a second panel of biomarkers is analyzed to diagnose the patient as having ATB or LTBI. The first panel of biomarkers to diagnose the patient as having TB can include analyzing expression levels of one or more, at least 2, at least 3, or at least 4, or at least 5, or at least 6, or at least 7, or at least 8, or at least 9, or at least 10, or at least 11 or more biomarkers. The second panel of biomarkers to diagnose the patient as having ATB or LTBI can include analyzing expression levels of one or more, at least 2, at least 3, or at least 4, or at least 5, or at least 6, or at least 7, or at least 8, or at least 9, or at least 10, or at least 11 or more biomarkers. In certain embodiments, the disclosure includes a first biomarker panel comprising from 1 to 6 (e.g., 1, 2, 3, 4, 5, or 6) biomarkers and a second biomarker panel comprising from 2 to 8 (e.g., 1, 2, 3, 4, 5, 6, 7, or 8) biomarkers. The first and second panel may share 1 or more biomarkers in common or there may be not overlap between the panels. For example, IL2 may be included in both panels.

In certain embodiments the combined set of biomarkers that are analyzed for expression level include IFN-γ, MIG, IL2, FOXP3, GBP1P1, VEGFA, and PLAU. The measurements for a first subset of the biomarkers, including IFN-γ, MIG, IL2, FOXP3, GBP1P1, and optionally VEGFA are analyzed to diagnose the patient as either having an MTB infection or being not infected and the measurements for a second subset of the biomarkers, including VEGFA, PLAU, and IL2 are analyzed to diagnose the patient as having ATB or LTBI. Optionally one or more markers selected from DUSP3, PLAU, SLP1, and PTGS2 may also be analyzed and included in the first subset of biomarkers. In some embodiments the expression levels of IFN-γ, IL2 and PLAU are measured and then the results for IFN-γ and IL2 are used to diagnose MTB infected vs. not infected and the results for IL2 and PLAU are used to diagnose ATB vs. LTBI.

In certain embodiments the combined set of biomarkers that are analyzed for expression level include IFN-γ, MIG, IL2, FOXP3, GBP1P1, GBP5, DUSP3, SLPI, VEGFA, and PLAU. The measurements for a first subset of the biomarkers, including IFN-γ, MIG, IL2, FOXP3, GBP1P1, and optionally VEGFA are analyzed to diagnose the patient as either having an MTB infection or being not infected and the measurements for a second subset of the biomarkers, including VEGFA, GBP1P1, GBP5, DUSP3, SLPI, PLAU, and optionally IL2 are analyzed to diagnose the patient as having ATB or LTBI. Optionally one or more markers selected from DUSP3, PLAU, SLP1, and PTGS2 may also be analyzed and included in the first subset of biomarkers. In some embodiments the expression levels of IFN-γ, IL2 and PLAU are measured and then the results for IFN-γ and IL2 are used to diagnose MTB infected vs. not infected and the results for IL2 and PLAU are used to diagnose ATB vs. LTBI.

In certain embodiments the combined set of biomarkers that are analyzed for expression level include IFN-γ, MIG, IL2, FOXP3, GBP1P1, GBP5, DUSP3, SLPI, VEGFA, and PLAU, and then the results for two or more of IFN-γ, MIG, IL2, FOXP3, GBP1P1, and VEGFA are used to diagnose MTB infected vs. not infected and the results for two or more of VEGFA, GBP1P1, GBP5, DUSP3, SLPI, PLAU, and IL2 are used to diagnose ATB vs. LTBI. For example, the expression levels of IFN-γ and MIG; IFN-γ, MIG and IL2; IFN-γ, MIG, IL2 and FOXP3; IFN-γ, MIG, IL2, FOXP3, and GBP1P1; or IFN-γ, MIG, IL2, and GBP1P1 can be used to diagnose MTB infected vs. not infected. The expression levels of VEGFA and GBP1P1; VEGFA, GBP1P1, and GBP5; VEGFA, GBP1P1, GBP5, and DUSP3; VEGFA, GBP1P1, GBP5, DUSP3, and SLPI; or VEGFA, GBP1P1, GBP5, DUSP3, SLPI, and PLAU; can be used to diagnose ATB vs. LTBI.

In some embodiments, a single assay is performed to measure the expression levels and calculate ΔCt values for each biomarker and the data is used to generate a value for a single gene expression level or each of multiple different gene expression levels (or gene signatures). Cut off values or “scores” from each signature may be used to diagnose the patient as not-infected versus TB infected or ATB vs LTBI. The cut-off values may be flexible depending on the setting, such as in populations with TB prevalence of 0.5% or higher, populations with structural risk factors for TB, populations with HIV or certain other health conditions (e.g., fibrotic lesion), populations with close contacts of individuals with TB disease, populations in prisons and penitentiary institutions, or populations in certain workplaces. The threshold for considering a result as TB infected, ATB, LTBI, or evaluation of risks (e.g., risk for progression to TB) may differ by setting. In some instances, flexible cut-offs can be utilized in an algorithm that combines results from two signatures to rule out ATB and initiate Tuberculosis Preventive Treatment (TPT) in patients with LTBI/Incipient TB, or triage patients by providing indication of Subclinical/Active TB to initiate full TB treatment.

In some embodiments a single assay is performed to measure the expression levels and calculate ΔCt values for each biomarker and the data is used to generate a value for each of two different gene signatures. A first cut off value or “score” from the first signature may be used to diagnose the patient as being infected or not and a second cut off value or score from the second signature may be used to diagnose the patient as having ATB or LTBI. The first signature used to diagnose the patient as being infected or not provides a result similar to conventional Interferon-Gamma Release Assays (IGRAs), and the second signature (ATB or LTBI) provides an estimate on where on the scale between stable LTBI and ATB the patient is and/or the risk for the patient to progress to ATB. Markers within the signature may be given different weights to calculate the “score”. If the same marker is included in both signatures it may be given the same or different weights in the calculation of the score for each signature. Additional clinical data such as risk assessment, radiography and other clinical and laboratory findings may also be incorporated into the determination of the score. In some aspects, the scores may be reported in different ways depending on the clinical setting. In some aspects the first and second scores may be differentially weighted depending on the clinical setting from which the sample was collected. For example, the analysis may vary depending on whether a clinician or a self-collected sample is used, and based on the availability of treatment and follow-up provided by the clinic. In some locations where variability in sample collection is high it may be beneficial to use an additional control gene to normalize or to compensate for variability in cell counts of the sample.

The methods described herein may be used to determine if a patient should receive preventative treatment for TB, a full-course of treatment for ATB or another treatment appropriate for the status of the patient's infection. For example, a patient is selected for treatment for tuberculosis if the patient has a diagnosis of ATB based on a biomarker expression profile as described herein. Patients with LTBI can progress to minimal TB, then subclinical TB and then to ATB through increased disease burden. Alternatively, patients can be treated and infection can be eliminated/quiescent. The methods described herein may be used to monitor progression and predict likelihood of progression to ATB for patients identified as having LTBI, or to predict/monitor treatment response to determine when infection has been eliminated/quiescent or when ATB has reverted to stable LTBI or has been eliminated/quiescent.

In one embodiment, the disclosure includes a method of treating a subject having ATB, the method comprising: diagnosing the subject with ATB according to a method described herein; and administering a therapeutically effective amount of at least one antibiotic to the subject if the subject has a positive tuberculosis diagnosis.

In another embodiment, the disclosure includes a method of treating a subject suspected of having an MTB infection, the method comprising: receiving information regarding the diagnosis of the subject according to a method described herein; and administering a therapeutically effective amount of at least one antibiotic to the subject if the patient has a positive MTB infection.

Antibiotics that may be used in treating tuberculosis are known in the art and include, but are not limited to, ethambutol, isoniazid, pyrazinamide, rifabutin, rifampicin, rifapentine, amikacin, capreomycin, cycloserine, ethionamide, levofloxacin, moxifloxacin, para-aminosalicylic acid, and streptomycin. Typically, several antibiotics are administered simultaneously to treat active tuberculosis, whereas typically a single antibiotic is administered to treat latent tuberculosis. Treatment may continue for at least a month or several months, up to one or two years, or longer, depending on whether the tuberculosis infection is active or latent. Longer treatment is generally required for severe tuberculosis infection, particularly if the infection becomes antibiotic resistant. Latent tuberculosis may be effectively treated in less time, typically 4 to 12 months, to prevent tuberculosis infection from becoming active. Subjects, whose infection is antibiotic resistant, may be screened to determine antibiotic sensitivity in order to identify antibiotics that will eradicate the tuberculosis infection. In addition, corticosteroid medicines also may be administered to reduce inflammation caused by active tuberculosis.

A recommended method to treat LTBI regardless of HIV status provides 6 to 9 months of daily isoniazid, or a 3 month regimen of weekly rifapentine plus isoniazid, or a 3 month regiment of daily isoniazid plus rifampicin. A 1 month regimen of daily rifapentine plus isoniazid or 4 months of daily rifampicin alone may also be provided as an alternative. In settings with high TB transmission, for example in adults and adolescents living with HIV who have an unknown or a positive LTBI test but not diagnosed with ATB disease, the patient may receive at least 36 months of daily isoniazid preventive therapy (IPT). Daily IPT for 36 months is recommended whether or not the patient is on antiretroviral treatment, and irrespective of the degree of immunosuppression, history of previous TB treatment and pregnancy in settings considered to have a high TB transmission as defined by national authorities. Other treatment preventive therapies (TPT) that are known in the art may also be used to treat patients diagnosed with LBTI by the methods provided herein. Different treatments may be recommended for each level of infection.

The methods of the disclosure, as described herein, can also be used for determining the prognosis of a subject and for monitoring treatment of a subject who has tuberculosis. A medical practitioner can monitor the progress of disease by measuring the levels of the biomarkers in biological samples from the patient.

The signatures may be used serially or in parallel, and the cut-off values may be modified depending on the need. For example, if the intent is to rule out ATB for TB preventive treatment or to rule in ATB for full-course TB treatment different cut-off values may be selected. In some instances of the methods being used in series, application of a first algorithm defining a cut-off value for a first signature can be used to distinguish infected from non-infected, followed by application of a second algorithm defining a cut-off value for a second signature that can be used to distinguish ATB from LTBI among those that are infected. If the first signature is negative or inconclusive (such as scoring below the cut-off) but the second signature is positive for ATB or LTBI, the second signature alone may be used for selecting treatment. In some instances of the methods being used in parallel, application of a single algorithm defining multiple cut-offs for each signature, that is, not infected, ATB, and LTBI is considered. In other instances of the methods being used in parallel, application of two different algorithms together, for example, a first algorithm defining a cut-off value to distinguish infected from non-infected, and a second algorithm defining a cut-off value to distinguish ATB vs. LTBI are considered. In further instances of the methods being used in parallel, application of two different algorithms together, for example, a first algorithm defining a cut-off value to distinguish ATB vs. non-ATB, and a second algorithm defining a cut-off value to distinguish LTBI vs. non-ATB are considered.

In some embodiments, the methods are used to monitor treatment response and provide an advantage over analyzing other previously known T-cell response markers. Preventive TB treatment response may potentially be predicted/monitored using for example FoxP3, which could be part of the MTB Infected signature, but in a separate channel and measured separately. Full-course TB treatment response could be monitored using the ATB vs. LTBI signature. A shift in the results of the signature analysis from the ATB group toward the LTBI group could be used as an indication of patient improvement. The signatures may also be useful in monitoring the efficiency of preventive TB treatment.

In some embodiments a combination of markers that are known T-cell response markers may be used for the MTB Infected signature, optionally with the addition of one or more additional marker identified herein, selected from DUSP3, PLAU, SLPI or PTGS2 to improve sensitivity.

In some embodiments the ATB vs. LTBI signature may be used to identify a patient as being at high risk of having subclinical TB/ATB even if the MTB signature is negative or inconclusive. In some studies, lower levels of IGNg expression have been correlated with poor patient outcome, so a patient negative for MTB based on the MTB Infected signature, but with a high score for the ATB vs. LTBI signature could be further evaluated for TB.

The methods described herein for prognosis or diagnosis of subjects who have tuberculosis may be used in individuals who have not yet been diagnosed (for example, preventative screening), or who have been diagnosed, or who are suspected of having tuberculosis (e.g., display one or more characteristic symptoms), or who are at risk of developing tuberculosis (e.g., have a genetic predisposition or presence of one or more developmental, environmental, or behavioral risk factors). For example, patients having one or more risk factors including, but not limited to, patients who are immunosuppressed, immunodeficient, elderly, suspected of having had exposure to a subject infected with tuberculosis, or having symptoms of lung disease may be screened by the methods described herein. The methods may also be used to detect latent or active tuberculosis infection or evaluate severity of disease. The methods may also be used to detect the response of tuberculosis to prophylactic or therapeutic treatments or other interventions. The methods can furthermore be used to help the medical practitioner in determining prognosis (e.g., worsening, status-quo, partial recovery, or complete recovery) of the patient, and the appropriate course of action, resulting in either further treatment or observation, or in discharge of the patient from the medical care center.

In one embodiment, the disclosure includes a method for distinguishing active tuberculosis from latent tuberculosis. The method comprises: obtaining a biological sample from a patient and measuring levels of expression of IFN-γ, MIG, IP10, IL2, FoxP3, PLAU, SLPI, VEGFA, DUSP3, GBP5, GBP1P1, ANKRD22, SERPING1, PTGS2, and IL10 biomarkers in the biological sample. The levels of expression of each biomarker are analyzed in conjunction with respective reference value ranges for each biomarker. Similarity of the levels of expression of the biomarkers to reference value ranges for a subject with active tuberculosis indicates that the patient has active tuberculosis, whereas similarity of the levels of expression of the biomarkers to reference value ranges for a subject with latent tuberculosis indicates that the patient has latent tuberculosis. Different combinations of biomarkers may be analyzed depending on variables including the desired time of antigen stimulation (e.g., at least 0.1 hour, 0.1-6 hours, 0.1-8 hours, 3-4 hours, 8-24 hours or 16-20 hours) and the type of clinic performing the testing.

In one embodiment, MIG, IFN-γ, GBP1P1, IL2, and optionally VEGFA are used in a signature to distinguish between MTB infected and not infected and VEGFA, GBP5, PLAU, DUSP3, GBP1P1, SLP1, and optionally IL2 are used in a signature to distinguish between ATB and LTBI.

In one embodiment, MIG, IFN-γ, GBP1P1, and IL2 are used in a signature to distinguish between MTB infected and not infected and VEGFA, GBP5, PLAU, DUSP3, GBP1P1, and SLP1 are used in a signature to distinguish between ATB and LTBI.

In one embodiment, the expression of only a single biomarker (such as MIG alone, IFN-γ alone, GBP1P1 alone, IL2 alone, or VEGFA alone) is used in a signature to distinguish between MTB infected and not infected and two or more of VEGFA, GBP5, PLAU, DUSP3, GBP1P1, SLP1, and optionally IL2 are used in a signature to distinguish between ATB and LTBI.

In one embodiment, the expression of two or more biomarkers selected from IFN-γ, MIG, IP10, IL2, FoxP3, PLAU, SLPI, VEGFA, DUSP3, GBP5, GBP1P1, ANKRD22, SERPING1, PTGS2, and IL10 are used in a signature to distinguish between MTB infected and not infected and one or more of VEGFA, GBP5, PLAU, DUSP3, GBP1P1, SLP1, and optionally IL2 are used in a signature to distinguish between ATB and LTBI.

In one embodiment, MIG, IFN-γ, IP10, and IL2 are used in a signature to distinguish between MTB infected and not infected and VEGFA, PLAU, IL2 and SLP1 are used in a signature to distinguish between ATB and LTBI.

In one embodiment, MIG, IFN-γ, FOXP3, GBP1P1, IL2 and optionally one or more of DUSP3, PLAU or SLP1 are used in a signature to distinguish between MTB infected and not infected and GBP5, PLAU, DUSP3, GBP1P1 and SLP1 are used in a signature to distinguish between ATB and LTBI.

In one embodiment, MIG, IFN-γ, GBP5, IL2 and either PLAU or SLP1 are used in a signature to distinguish between MTB infected and not infected and GBP5, PLAU, IL2 and SLP1 are used in a signature to distinguish between ATB and LTBI.

In one embodiment, VEGFA, PLAU, IL2 and SLP1 are used in a signature to monitor treatment response of ATB patients.

Biomarker data may be analyzed by a variety of methods to identify biomarkers and determine the statistical significance of differences in observed levels of expression of the biomarkers between test and reference expression profiles in order to evaluate whether a patient has latent or active tuberculosis or some other pulmonary or infectious disease. In certain embodiments, patient data is analyzed by one or more methods including, but not limited to, multivariate linear discriminant analysis (LDA), receiver operating characteristic (ROC) analysis, principal component analysis (PCA), random forest, support vector machines, elastic net methods, ensemble data mining methods, significance analysis of microarrays (SAM), cell specific significance analysis of microarrays (csSAM), spanning-tree progression analysis of density-normalized events (SPADE), and multi-dimensional protein identification technology (MUDPIT) analysis. (See, e.g., Hilbe (2009) Logistic Regression Models, Chapman & Hall/CRC Press; McLachlan (2004) Discriminant Analysis and Statistical Pattern Recognition. Wiley Interscience; Zweig et al. (1993) Clin. Chem. 39:561-577; Breiman (2001) Random forests, Machine Learning 45:5032; Pepe (2003) The statistical evaluation of medical tests for classification and prediction, New York, N.Y.: Oxford; Sing et al. (2005) Bioinformatics 21:3940-3941; Tusher et al. (2001) Proc. Natl. Acad. Sci. U.S.A. 98:5116-5121; Oza (2006) Ensemble data mining, NASA Ames Research Center, Moffett Field, Calif., USA; English et al. (2009) J. Biomed. Inform. 42(2):287-295; Zhang (2007) Bioinformatics 8: 230; Shen-Orr et al. (2010) Journal of Immunology 184:144-130; Qiu et al. (2011) Nat. Biotechnol. 29(10):886-891; Ru et al. (2006) J. Chromatogr. A. 1111(2): 166-174, Jolliffe Principal Component Analysis (Springer Series in Statistics, 2.sup.nd edition, Springer, N Y, 2002), Koren et al. (2004) IEEE Trans Vis Comput Graph 10:459-470; herein incorporated by reference in their entireties.)

The present assay relies on the polymerase chain reaction (PCR) and can be carried out in a substantially automated manner using a commercially available nucleic acid amplification system. Exemplary nonlimiting nucleic acid amplification systems that can be used to carry out the methods of the disclosure include the GENEXPERT® system, a GENEXPERT® Infinity system, and GENEXPERT® Xpress System (Cepheid, Sunnyvale, Calif.). The amplification system may be available at the same location as the individual to be tested, such as a health care provider's office, a clinic or a community hospital, so processing is not delayed by a requirement to transport the sample to another facility. The present assay can be completed in under 3 hours, in some embodiments, under 2 hours, in some embodiments, under 1 hour, in some embodiments, under 45 minutes, in some embodiments, under 35 minutes, and in some embodiments, under 30 minutes, using an automated system, for example, the GENEXPERT® system.

### General Methods

Compositions and methods for measuring the expression levels of genes in a sample and diagnosing tuberculosis are provided. In some embodiments, compositions and methods for distinguishing between ATB and LTBI are provided.

In some embodiments, a method of detecting ATB or LTBI in a subject further comprises detecting at least one endogenous control, such as a sample adequacy control (SAC). In some embodiments, a method of detecting tuberculosis in a subject further comprises detecting at least one exogenous control, such as a sample processing control (SPC). In some embodiments, the SPC is an RNA control. In some embodiments, the SPC is ARMORED® RNA.

In the present disclosure, the terms “target RNA” and “target gene” are used interchangeably to refer any of the biomarker genes described herein, as well as to exogenous and/or endogenous controls. Thus, it is to be understood that when a discussion is presented in terms of a target gene, that discussion is specifically intended to encompass the biomarker genes, any endogenous control(s) (e.g., SAC), and any exogenous control(s) (e.g., SPC).

In some embodiments, the expression level of the biomarker genes is detected in a blood sample. In some embodiments, a target gene is detected in a sample to which a buffer (such as a preservative) has been added. In some embodiments, the presence of the biomarker genes is detected in a blood sample that has been incubated in the presence of one or more MTB antigens.

In some embodiments the blood is drawn from the patient using lithium-heparin tubes and the resulting heparinized whole blood is then incubated in a tube with one or more antigens for stimulation. The incubation may be at room temperature or greater, and includes incubation at between 35° C. and 39° C. for a period of time, for example, at least 0.1 hour, 0.1-8 hours, 0.1-4 hours, 1-6 hours, 2-3 hours, 3-4 hours, 3-6 hours, overnight (8-20 hours, 12-20 hours, or 16-20 hours) or up to 24 hours depending on the desired workflow. Antigen stimulated blood is added directly to the GeneXpert® cartridge. A 3-4 hour incubation may be preferred if it is desirable to complete the assay in a single day. A 16-20 hour incubation may be preferred if the test is to be performed overnight. In some embodiments the sample may be stored for hours, days, or a week after stimulation and prior to analysis. The sample may be stored for longer than a week (such as a month or longer) if frozen. In some aspects the incubation may be performed in a tube that is specially designed to attach to the cartridge that will be used for performing some of the sample prep steps or may be part of the cartridge.

Both short (e.g., greater than 0 hours to less than 8 hours, 1-7 hours, 2-6 hours, 1-4 hours or 2-3 hours) and long (e.g., 8-24 hours, 8-20 hours, 8-16 hours or 16-20 hours) incubation of the blood with antigens can be used in the methods disclosed herein. The inventors have found that in some instances, the first signature which determines tuberculosis infected vs. not infected may perform slightly better at long incubation, while the second signature which determines ATB vs. LTBI may perform better at the short incubation time. Changes in the incubation time may be demonstrated by slight changes in the data generated from the assay, such as Ct values for the biomarker in PCR detection. Therefore, depending on the incubation time or other changes in the assay, the disclosed methods may be optimized to use different assay protocols. In some embodiments, the methods can be optimized to have slightly different cycling conditions (e.g., temperature, flow volumes and rates, and incubation times), target genes (e.g., different combinations of targets in the assay, different weighting of the targets in the assay, or both), primers, probes, among other changes in assay protocols for short and for long incubation times, which may require a different data-analysis algorithm.

In the methods, kits, and systems provided herein, changes in the assay protocol (such as cycling conditions, target gene, reference method, short or long incubation times) may have an associated unique assay definition file. As used herein, the term assay definition file (ADF) refers to a file that provides at least some, and typically all of the assay specific parameters for that workflow. For example, the ADF can contain sample prep parameters (for example, incubation time), PCR protocols, scripts and thresholds used to generate results, and parameters used to drive the data analysis engine within the instrument. Depending on the assay protocol, each biomarker may be differentially weighted based on the signature being determined in an ADF. In some examples, the cartridges provided herein can have at least two (2) ADFs with slightly different signature algorithms (for e.g., markers and/or coefficients/weighting) for short and long incubation times. In some instances, however, the signature algorithms for short and for long incubation times exhibit only slight to no differences such that a single ADF is compatible with all the workflows. Therefore, the cartridges provided herein can have a single ADF for short and long incubation times.

In some embodiments, the detecting of the gene expression level of each biomarker is done quantitatively. In other embodiments, the detecting is done qualitatively. In some embodiments, detecting a target gene comprises forming a complex comprising a polynucleotide and a nucleic acid selected from a target gene, a cDNA reverse transcribed from a target gene, a DNA amplicon of a target gene, and a complement of a target gene. In some embodiments, detecting a target gene comprises RT-PCR. In some embodiments, detecting a target gene comprises quantitative RT-PCR or real-time RT-PCR. In some embodiments, a sample adequacy control (SAC) and/or a sample processing control (SPC) is detected in the same assay as the target gene. In some embodiments, expression levels are normalized to another biomarker with an expression level that is not expected to change in response to MTB infection and TB antigen stimulation, for example, a housekeeping gene like TBP (TATA-box binding protein) or a T-cell marker like CD3E (cluster of differentiation 3).

In some embodiments, the presence of the biomarker genes can be measured in samples collected at one or more times from a subject to monitor treatment for tuberculosis or Latent TB in the subject. In some embodiments, the assay may be used in a subject suspected of respiratory tract infection, e.g., after consultation with their healthcare provider. In some embodiments, the present assay may be used as part of routine and/or preventative healthcare for a subject. In some embodiments, the present assay may be used seasonally as part of routine and/or preventative healthcare for a subject. In some embodiments, the present assay may be used as part of routine and/or preventative healthcare for subjects who are at particular risk from tuberculosis.

In some embodiments, less than 5 ml, less than 4 ml, less than 3 ml, less than 2 ml, less than 1 ml, or less than 0.75 ml of sample or buffered sample are used in the present methods. In some embodiments, 0.1 ml to 1 ml of sample or buffered sample is used in the present methods.

The clinical sample to be tested is, in some embodiments, fresh (i.e., never frozen). In other embodiments, the sample is a frozen specimen. Frozen specimens may be mixed with stabilizing agents or lysis buffer before being frozen. In some embodiments, the sample is a tissue sample, such as a formalin-fixed paraffin embedded sample. In some embodiments, the sample is a liquid cytology sample.

In some embodiments, the sample to be tested is obtained from an individual who has one or more symptoms of tuberculosis.

In some embodiments, methods described herein can be used for routine screening of healthy individuals with no risk factors. In some embodiments, methods described herein are used to screen asymptomatic individuals, for example, during routine or preventative care. In some embodiments, methods described herein are used to screen women who are pregnant or who are attempting to become pregnant. In some embodiments the method is used to test patients prior to immunosuppressive therapy.

In some embodiments, the methods described herein can be used to assess the effectiveness of a treatment for tuberculosis infection in a patient.

In some embodiments, information concerning the diagnosis of tuberculosis in the subject is communicated to a medical practitioner. A “medical practitioner,” as used herein, refers to an individual or entity that diagnoses and/or treats patients, such as a hospital, a clinic, a physician's office, a physician, a nurse, or an agent of any of the aforementioned entities and individuals. In some embodiments, the methods are carried out at a laboratory that has received the subject's sample from the medical practitioner or agent of the medical practitioner. The laboratory carries out the detection by any method, including those described herein, and then communicates the results to the medical practitioner. A result is “communicated,” as used herein, when it is provided by any means to the medical practitioner. In some embodiments, such communication may be oral or written, may be by telephone, in person, by e-mail, by mail or other courier, or may be made by directly depositing the information into, e.g., a database accessible by the medical practitioner, including databases not controlled by the medical practitioner. In some embodiments the result of the assay is combined with clinical parameters, data, or information about other risk factors, for example a chest x-ray, to make a diagnosis. In some embodiments, the information is maintained in electronic form. In some embodiments, the information can be stored in a memory or other computer readable medium, such as RAM, ROM, EEPROM, flash memory, computer chips, digital video discs (DVD), compact discs (CDs), hard disk drives (HDD), magnetic tape, etc. The results may also be provided using a web based application that may be provided to the health care practitioner or to the patient on a smart phone or other mobile device. In some aspects, results may be provided to the patient via a mobile device.

In some embodiments, the method further comprises receiving a communication from the laboratory that indicates the diagnosis of tuberculosis in the sample. A “laboratory,” as used herein, is any facility that detects the target gene in a sample by any method, including the methods described herein, and communicates the result to a medical practitioner. In some embodiments, a laboratory is under the control of a medical practitioner. In some embodiments, a laboratory is not under the control of the medical practitioner.

As used herein, when a method relates to diagnosing tuberculosis infection, the method includes activities in which the steps of the method are carried out, but the result is negative for the presence of tuberculosis infection. That is, detecting, determining, monitoring, and diagnosing tuberculosis infection include instances of carrying out the methods that result in either positive or negative results. In some embodiments, detecting, determining, monitoring, and diagnosing tuberculosis infection include instances of carrying out the methods to determine a risk score instead of a positive or negative result. For example, clinical data such as symptoms, chest X-ray, bacteriological test results and IGRA test results may be incorporated into the algorithm used as the reference methods. Thus, the cut off value or “score” from each signature measured can vary depending on the sensitivity and specificity of the reference methods. In other instances, the positive predictive value of the test method can depend on the specific population, setting, and therapeutic status of the subject, or variability in sample collection method. For example, it has been observed that while sensitivity of the WHO 4-symptom TB screening rule is about 89% among antiretroviral therapy (ART)-naive people living with HIV, it is only 51% among people on ART, due to a higher prevalence of subclinical TB among stable ART patients. Therefore, use of a cut off value or risk-score can be used to modulate the sensitivity or specificity of the results based on variables in the subject population or clinical setting. Differentiation of results by risk-score cutoff allows flexibility in designing differentiated TB care to maximize impact of available resources.

In some embodiments, at least one endogenous control (e.g., an SAC) and/or at least one exogenous control (e.g., an SPC) are detected simultaneously with the biomarkers in a single reaction. In some embodiments, at least one exogenous control (e.g., an SPC) is detected simultaneously with the biomarkers in a single reaction.

### Exemplary Controls

In some embodiments, a normal level (a “control”) of a target RNA, can be determined as an average level or range that is characteristic of a healthy sample or other reference material, against which the level measured in the sample can be compared. The determined average or range of a target RNA in normal subjects can be used as a benchmark for detecting above-normal levels of the target RNA that are indicative of TB. In some embodiments, normal levels of a target RNA can be determined using individual or pooled RNA-containing samples from one or more individuals, such as blood from healthy individuals.

In some embodiments, an assay described herein comprises detecting the biomarkers and at least one endogenous control. In some embodiments, the endogenous control is a sample adequacy control (SAC). In some such embodiments, if none of the biomarkers are detected in a sample, and the SAC is also not detected in the sample, the assay result is considered “invalid” because the sample may have been insufficient. While not intending to be bound by any particular theory, an insufficient sample may be too dilute, contain too little cellular material, contain an assay inhibitor, etc. In some embodiments, the failure to detect an SAC may indicate that the assay reaction failed. In some embodiments, an endogenous control is an RNA (such as an mRNA, tRNA, ribosomal RNA, etc.). Nonlimiting exemplary endogenous controls include ABL mRNA, GUSB mRNA, GAPDH mRNA, TUBB mRNA, and UPK1a mRNA. Other endogenous controls that may be used in connection with the present methods include CD3e, TBP, CD4 and B2M.

In some embodiments, an exogenous control (such as an SPC) is added during performance of an assay, such as with one or more buffers or reagents. In some embodiments, when a GENEXPERT® system is to be used, the SPC is included in the GENEXPERT® cartridge. In some embodiments, an exogenous control (such as an SPC) is an ARMORED® RNA, which is protected by a bacteriophage coat.

In some embodiments, an endogenous control and/or an exogenous control is detected contemporaneously, such as in the same assay, as detection of the biomarkers. In some embodiments, an assay comprises reagents for detecting the biomarkers and an exogenous control simultaneously in the same assay reaction. In some such embodiments, for example, an assay reaction comprises a primer set for amplifying each of the biomarkers, and a primer set for amplifying an exogenous control, and labeled probes for detecting the amplification products (such as, for example, TAQMAN® probes).

In some embodiments, the level of a target RNA is normalized to an endogenous control RNA. Normalization may comprise, for example, determination of the difference of the level of the target RNA to the level of the endogenous control RNA. In some such embodiments, the level of the RNAs are represented by a Ct value obtained from quantitative PCR. In some such embodiments, the difference between two measurements is expressed as ΔCt. ΔCt may be calculated as Ct[target RNA]-Ct[endogenous control] or Ct[endogenous control]-Ct[target RNA]. In certain embodiments, ΔCt=Ct[endogenous control]-Ct[biomarker]. In some embodiments, a threshold ΔCt value is set, above or below which a particular diagnosis is indicated. In some such embodiments, the ΔCt threshold is set as the ΔCt value below which 75% of normal samples are correctly characterized. Different thresholds may be applicable to different assays so in some the threshold may be higher, 80%, 90%, 95%, or 97% for example and in some the threshold may be lower, 50%, 60%, or 70% for example. In some such embodiments, a ΔCt value that is higher than the threshold ΔCt value is indicative of a particular disease diagnosis.

In some embodiments, a threshold Ct (or a “cutoff Ct”) value for a target RNA, that is indicative of TB, ATB or LTBI, has previously been determined. In such embodiments, a control sample may not be assayed concurrently with the test sample. In some embodiments, as discussed herein, a ΔCt threshold value is determined, above which TB is indicated or which differentiates between ATB and LTBI, has previously been determined.

In some embodiments, linear discriminant analysis (LDA) is used, for example, to combine two or more of the markers into a single combined scale. In some such embodiments, a single threshold value is used for the markers included in the LDA for each of the signatures, e.g. there is a separate threshold value for each set of markers or each signature analysis.

### Exemplary RNA Preparation

Target RNA can be prepared by any appropriate method. Total RNA can be isolated by any method, including, but not limited to, the protocols set forth in Wilkinson, M. (1988) Nucl. Acids Res. 16(22):10,933; and Wilkinson, M. (1988) Nucl. Acids Res. 16(22): 10934, or by using commercially-available kits or reagents, such as the TRIzol® reagent (Invitrogen), Total RNA Extraction Kit (iNtRON Biotechnology), Total RNA Purification Kit (Norgen Biotek Corp.), RNAqueous™ (Invitrogen), MagMAX™ (Applied Biosystems), RecoverAll™ (Invitrogen), RNAeasy (Qiagen), etc.

In some embodiments, RNA levels are measured in a sample in which RNA has not first been purified from the cells. In some such embodiments, the cells are subject to a lysis step to release the RNA. Nonlimiting exemplary lysis methods include sonication (for example, for 2-15 seconds, 8-18 μm at 36 kHz); chemical lysis, for example, using a detergent; and various commercially available lysis reagents (such as RNAeasy lysis buffer, Qiagen). In some embodiments, RNA levels are measured in a sample in which RNA has been isolated.

In some embodiments, RNA is modified before a target RNA is detected. In some embodiments, all of the RNA in the sample is modified. In some embodiments, just the particular target RNAs to be analyzed are modified, e.g., in a sequence-specific manner. In some embodiments, RNA is reverse transcribed. In some such embodiments, RNA is reverse transcribed using a reverse transcriptase enzyme such as MMLV, AMV or variants thereof that have been engineered to have features such as reduced RNAse H activity and increased processivity, sensitivity, and thermostability. Nonlimiting exemplary conditions for reverse transcribing RNA using MMLV reverse transcriptase include incubation from 5 to 20 minutes at 40° C. to 50° C.

When a target RNA is reverse transcribed, a DNA complement of the target RNA is formed. In some embodiments, the complement of a target RNA is detected rather than a target RNA itself (or a DNA copy of the RNA itself). Thus, when the methods discussed herein indicate that a target RNA is detected, or the level of a target RNA is determined, such detection or determination may be carried out on a complement of a target RNA instead of, or in addition to, the target RNA itself. In some embodiments, when the complement of a target RNA is detected rather than the target RNA, a polynucleotide for detection is used that is complementary to the complement of the target RNA. In some such embodiments, a polynucleotide for detection comprises at least a portion that is identical in sequence to the target RNA, although it may contain thymidine in place of uridine, and/or comprise other modified nucleotides.

### Exemplary Analytical Methods

Any analytical procedure capable of permitting specific detection of a target gene may be used in the methods herein presented. Exemplary nonlimiting analytical procedures include, but are not limited to, nucleic acid amplification methods, PCR methods, isothermal amplification methods, and other analytical detection methods known to those skilled in the art.

In some embodiments, the method of detecting a target gene comprises amplifying the gene and/or a complement thereof. Such amplification can be accomplished by any method. Exemplary methods include, but are not limited to, isothermal amplification, real time RT-PCR, endpoint RT-PCR, and amplification using T7 polymerase from a T7 promoter annealed to a DNA, such as provided by the SenseAmp Plus™ Kit available at Implen, Germany.

When a target gene is amplified, in some embodiments, an amplicon of the target gene is formed. An amplicon may be single stranded or double-stranded. In some embodiments, when an amplicon is single-stranded, the sequence of the amplicon is related to the target gene in either the sense or antisense orientation. In some embodiments, an amplicon of a target gene is detected rather than the target gene itself. Thus, when the methods discussed herein indicate that a target gene is detected, such detection may be carried out on an amplicon of the target gene instead of, or in addition to, the target gene itself. In some embodiments, when the amplicon of the target gene is detected rather than the target gene, a polynucleotide for detection is used that is complementary to the complement of the target gene. In some embodiments, when the amplicon of the target gene is detected rather than the target gene, a polynucleotide for detection is used that is complementary to the target gene. Further, in some embodiments, multiple polynucleotides for detection may be used, and some polynucleotides may be complementary to the target gene and some polynucleotides may be complementary to the complement of the target gene.

In some embodiments, the method of detecting a target gene comprises PCR, as described below. In some embodiments, detecting one or more target genes comprises real-time monitoring of a PCR reaction, which can be accomplished by any method. Such methods include, but are not limited to, the use of TAQMAN®, molecular beacons, or Scorpions probes (i.e., energy transfer (ET) probes, such as FRET probes) and the use of intercalating dyes, such as SYBR green, EvaGreen, thiazole orange, YO-PRO, TO-PRO, etc.

Nonlimiting exemplary conditions for amplifying a cDNA that has been reverse transcribed from the target RNA are as follows. An exemplary cycle comprises an initial denaturation at 90° C. to 100° C. for 20 seconds to 5 minutes, followed by cycling that comprises denaturation at 90° C. to 100° C. for 1 to 10 seconds, followed by annealing and amplification at 60° C. to 75° C. for 10 to 40 seconds. A further exemplary cycle comprises 20 seconds at 94° C., followed by up to 3 cycles of 1 second at 95° C., 35 seconds at 62° C., 20 cycles of 1 second at 95° C., 20 seconds at 62° C., and 14 cycles of 1 second at 95° C., 35 seconds at 62° C. In some embodiments, for the first cycle following the initial denaturation step, the cycle denaturation step is omitted. In some embodiments, Taq polymerase is used for amplification. In some embodiments, the cycle is carried out at least 10 times, at least 15 times, at least 20 times, at least 25 times, at least 30 times, at least 35 times, at least 40 times, or at least 45 times. In some embodiments, Taq is used with a hot start function. In some embodiments, the amplification reaction occurs in a GENEXPERT® cartridge, and amplification of the target genes and an exogenous control occurs in the same reaction. In some embodiments, detection of the target genes occurs in less than 3 hours, less than 2.5 hours, less than 2 hours, less than 1 hour, less than 45 minutes, less than 40 minutes, less than 35 minutes, or less than 30 minutes from initial denaturation through the last extension.

In some embodiments, detection of a target gene comprises forming a complex comprising a polynucleotide that is complementary to a target gene or to a complement thereof, and a nucleic acid selected from the target gene, a DNA amplicon of the target gene, and a complement of the target gene. Thus, in some embodiments, the polynucleotide forms a complex with a target gene. In some embodiments, the polynucleotide forms a complex with a complement of the target RNA, such as a cDNA that has been reverse transcribed from the target RNA. In some embodiments, the polynucleotide forms a complex with a DNA amplicon of the target gene. When a double-stranded DNA amplicon is part of a complex, as used herein, the complex may comprise one or both strands of the DNA amplicon. Thus, in some embodiments, a complex comprises only one strand of the DNA amplicon. In some embodiments, a complex is a triplex and comprises the polynucleotide and both strands of the DNA amplicon. In some embodiments, the complex is formed by hybridization between the polynucleotide and the target gene, complement of the target gene, or DNA amplicon of the target gene. The polynucleotide, in some embodiments, is a primer or probe.

In some embodiments, a method comprises detecting the complex. In some embodiments, the complex does not have to be associated at the time of detection. That is, in some embodiments, a complex is formed, the complex is then dissociated or destroyed in some manner, and components from the complex are detected. An example of such a system is a TAQMAN® assay. In some embodiments, when the polynucleotide is a primer, detection of the complex may comprise amplification of the target gene, a complement of the target gene, or a DNA amplicon of the target gene.

In some embodiments the analytical method used for detecting at least one target gene in the methods set forth herein includes real-time quantitative PCR. In some embodiments, the analytical method used for detecting at least one target gene includes the use of a TAQMAN® probe. The assay uses energy transfer (“ET”), such as fluorescence resonance energy transfer (“FRET”), to detect and quantitate the synthesized PCR product. Typically, the TAQMAN® probe comprises a fluorescent dye molecule coupled to the 5′-end and a quencher molecule coupled to the 3′-end, such that the dye and the quencher are in close proximity, allowing the quencher to suppress the fluorescence signal of the dye via FRET. When the polymerase replicates the chimeric amplicon template to which the TAQMAN® probe is bound, the 5′-nuclease of the polymerase cleaves the probe, decoupling the dye and the quencher so that the dye signal (such as fluorescence) is detected. Signal (such as fluorescence) increases with each PCR cycle proportionally to the amount of probe that is cleaved.

In some embodiments, a target gene is considered to be detected if any signal is generated from the TAQMAN® probe during the PCR cycling. For example, in some embodiments, if the PCR includes 40 cycles, if a signal is generated at any cycle during the amplification, the target gene is considered to be present and detected. In some embodiments, if no signal is generated by the end of the PCR cycling, the target gene is considered to be absent and not detected.

In some embodiments, quantitation of the results of real-time PCR assays is done by constructing a standard curve from a nucleic acid of known concentration and then extrapolating quantitative information for target genes of unknown concentration. In some embodiments, the nucleic acid used for generating a standard curve is a DNA (for example, an endogenous control, or an exogenous control). In some embodiments, the nucleic acid used for generating a standard curve is a purified double-stranded plasmid DNA or a single-stranded DNA generated in vitro.

In some embodiments, in order for an assay to indicate MTB infection, ATB or LTBI, the Ct values for an endogenous control (such as an SAC) and/or an exogenous control (such as an SPC) must be within a previously-determined valid range. That is, in some embodiments, the absence of TB cannot be confirmed unless the controls are detected, indicating that the assay was successful. In some embodiments, the assay includes an exogenous control. Ct values are inversely proportional to the amount of nucleic acid target in a sample.

In some embodiments, a threshold Ct (or a “cutoff Ct”) value for a target gene (including an endogenous control and/or exogenous control), below which the gene is considered to be detected, has previously been determined. In some embodiments, a threshold Ct is determined using substantially the same assay conditions and system (such as a GENEXPERT®) on which the samples will be tested. In some embodiments a ΔCt value is determined

In addition to the TAQMAN® assays, other real-time PCR chemistries useful for detecting and quantitating PCR products in the methods presented herein include, but are not limited to, Molecular Beacons, Scorpions probes and intercalating dyes, such as SYBR Green, EvaGreen, thiazole orange, YO-PRO, TO-PRO, etc., which are discussed below.

In various embodiments, real-time PCR detection is utilized to detect, in a single multiplex reaction, the biomarkers, and optionally an endogenous control, and an exogenous control. In some multiplex embodiments, a plurality of probes, such as TAQMAN® probes, each specific for a different target, is used. In some embodiments, each target gene-specific probe is spectrally distinguishable from the other probes used in the same multiplex reaction. A nonlimiting exemplary seven-color multiplex system is described, e.g., in Lee et al., BioTechniques, 27: 342-349 and a ten-color multiplex system has been described, e.g., in Xie et al. N Engl J Med 2017; 377:1043-1054 and Chakravorty et al. J Clin Microbiol 2016; 55:183-198.

In some embodiments, quantitation of real-time RT PCR products is accomplished using a dye that binds to double-stranded DNA products, such as SYBR Green, EvaGreen, thiazole orange, YO-PRO, TO-PRO, etc. In some embodiments, the assay is the QuantiTect SYBR Green PCR assay from Qiagen. In this assay, total RNA is first isolated from a sample. Total RNA is subsequently poly-adenylated at the 3′-end and reverse transcribed using a universal primer with poly-dT at the 5′-end. In some embodiments, a single reverse transcription reaction is sufficient to assay multiple target RNAs. Real-time RT-PCR is then accomplished using target RNA-specific primers and an miScript Universal Primer, which comprises a poly-dT sequence at the 5′-end. SYBR Green dye binds non-specifically to double-stranded DNA and upon excitation, emits light. In some embodiments, buffer conditions that promote highly-specific annealing of primers to the PCR template (e.g., available in the QuantiTect SYBR Green PCR Kit from Qiagen) can be used to avoid the formation of non-specific DNA duplexes and primer dimers that will bind SYBR Green and negatively affect quantitation. Thus, as PCR product accumulates, the signal from SYBR Green increases, allowing quantitation of specific products.

Real-time PCR is performed using any PCR instrumentation available in the art. Typically, instrumentation used in real-time PCR data collection and analysis comprises a thermal cycler, optics for fluorescence excitation and emission collection, and optionally a computer and data acquisition and analysis software.

In some embodiments, detection and/or quantitation of real-time PCR products is accomplished using a dye that binds to double-stranded DNA products, such as SYBR Green, EvaGreen, thiazole orange, YO-PRO, TO-PRO, etc. In some embodiments, the analytical method used in the methods described herein is a DASL® (DNA-mediated Annealing, Selection, Extension, and Ligation) Assay. In some embodiments, total RNA is isolated from a sample to be analyzed by any method. Total RNA may then be polyadenylated (>18 A residues are added to the 3′-ends of the RNAs in the reaction mixture). The RNA is reverse transcribed using a biotin-labeled DNA primer that comprises from the 5′ to the 3′ end, a sequence that includes a PCR primer site and a poly-dT region that binds to the poly-dA tail of the sample RNA. The resulting biotinylated cDNA transcripts are then hybridized to a solid support via a biotin-streptavidin interaction and contacted with one or more target RNA-specific polynucleotides. The target RNA-specific polynucleotides comprise, from the 5′-end to the 3′-end, a region comprising a PCR primer site, region comprising an address sequence, and a target RNA-specific sequence.

In some DASL® embodiments, the target RNA-specific sequence comprises at least 8, at least 9, at least 10, at least 11, at least 12, at least 13, at least 14, at least 15, at least 16, at least 17, at least 18, at least 19 contiguous nucleotides having a sequence that is the same as, or complementary to, at least 8, at least 9, at least 10, at least 11, at least 12, at least 13, at least 14, at least 15, at least 16, at least 17, at least 18, at least 19 contiguous nucleotides of a target RNA, an endogenous control RNA, or an exogenous control RNA.

After hybridization, the target RNA-specific polynucleotide is extended, and the extended products are then eluted from the immobilized cDNA array. A second PCR reaction using a fluorescently-labeled universal primer generates a fluorescently-labeled DNA comprising the target RNA-specific sequence. The labeled PCR products are then hybridized to a microbead array for detection and quantitation.

In some embodiments, the analytical method used for detecting and quantifying the target genes in the methods described herein is a bead-based flow cytometric assay. See Lu J. et al. (2005) Nature 435:834-838, which is incorporated herein by reference in its entirety. An example of a bead-based flow cytometric assay is the xMAP® technology of Luminex, Inc. See luminexcorp.com. In some embodiments, total RNA is isolated from a sample and is then labeled with biotin. The labeled RNA is then hybridized to target RNA-specific capture probes (e.g., FlexmiR™ products sold by Luminex, Inc. at luminexcorp.com) that are covalently bound to microbeads, each of which is labeled with 2 dyes having different fluorescence intensities. A streptavidin-bound reporter molecule (e.g., streptavidin-phycoerythrin, also known as “SAPE”) is attached to the captured target RNA and the unique signal of each bead is read using flow cytometry. In some embodiments, the RNA sample is first polyadenylated, and is subsequently labeled with a biotinylated 3DNA™ dendrimer (i.e., a multiple-arm DNA with numerous biotin molecules bound thereto), using a bridging polynucleotide that is complementary to the 3′-end of the poly-dA tail of the sample RNA and to the 5′-end of the polynucleotide attached to the biotinylated dendrimer. The streptavidin-bound reporter molecule is then attached to the biotinylated dendrimer before analysis by flow cytometry. In some embodiments, biotin-labeled RNA is first exposed to SAPE, and the RNA/SAPE complex is subsequently exposed to an anti-phycoerythrin antibody attached to a DNA dendrimer, which can be bound to as many as 900 biotin molecules. This allows multiple SAPE molecules to bind to the biotinylated dendrimer through the biotin-streptavidin interaction, thus increasing the signal from the assay.

In some embodiments, the analytical method used for detecting and quantifying the levels of the at least one target gene in the methods described herein is by gel electrophoresis and detection with labeled probes (e.g., probes labeled with a radioactive or chemiluminescent label), such as by northern blotting. In some embodiments, total RNA is isolated from the sample, and then is size-separated by SDS polyacrylamide gel electrophoresis. The separated RNA is then blotted onto a membrane and hybridized to radiolabeled complementary probes. In some embodiments, exemplary probes contain one or more affinity-enhancing nucleotide analogs as discussed below, such as locked nucleic acid (“LNA”) analogs, which contain a bicyclic sugar moiety instead of deoxyribose or ribose sugars. See, e.g., Várallyay, E. et al. (2008) Nature Protocols 3(2):190-196, which is incorporated herein by reference in its entirety.

In some embodiments, detection and quantification of one or more target genes is accomplished using microfluidic devices and single-molecule detection. In some embodiments, target RNAs in a sample of isolated total RNA are hybridized to two probes, one which is complementary to nucleic acids at the 5′-end of the target RNA and the second which is complementary to the 3′-end of the target RNA. Each probe comprises, in some embodiments, one or more affinity-enhancing nucleotide analogs, such as LNA nucleotide analogs and each is labeled with a different fluorescent dye having different fluorescence emission spectra (i.e., detectably different dyes). The sample is then flowed through a microfluidic capillary in which multiple lasers excite the fluorescent probes, such that a unique coincident burst of photons identifies a particular target RNA, and the number of particular unique coincident bursts of photons can be counted to quantify the amount of the target RNA in the sample. In some alternative embodiments, a target RNA-specific probe can be labeled with 3 or more distinct labels selected from, e.g., fluorophores, electron spin labels, etc., and then hybridized to an RNA sample.

### Exemplary Automation and Systems

In some embodiments, gene expression is detected using an automated sample handling and/or analysis platform. In some embodiments, commercially available automated analysis platforms are utilized. For example, in some embodiments, the GENEXPERT® system (Cepheid, Sunnyvale, Calif.) is utilized.

The present disclosure is illustrated for use with the GENEXPERT® system. Exemplary sample preparation and analysis methods are described below. However, the present disclosure is not limited to a particular detection method or analysis platform. One of skill in the art recognizes that any number of platforms and methods may be utilized.

The GENEXPERT® utilizes a self-contained, single use cartridge. Sample extraction, amplification, and detection may all carried out within this self-contained “laboratory in a cartridge.” (See e.g., U.S. Pat. Nos. 5,958,349, 6,403,037, 6,440,725, 6,783,736, 6,818,185; each of which is herein incorporated by reference in its entirety.)

Components of the cartridge include, but are not limited to, processing chambers containing reagents, filters, and capture technologies useful to extract, purify, and amplify target nucleic acids. A valve enables fluid transfer from chamber to chamber and contain nucleic acids lysis and filtration components. An optical window enables real-time optical detection. A reaction tube enables very rapid thermal cycling.

In some embodiments, the GENEXPERT® system includes a plurality of modules for scalability. Each module includes a plurality of cartridges, along with sample handling and analysis components.

After the sample is added to the cartridge, the sample is contacted with lysis buffer and released nucleic acid (NA) is bound to an NA-binding substrate such as a silica or glass substrate. The sample supernatant is then removed, and the NA eluted in an elution buffer such as a Tris/EDTA buffer. The eluate may then be processed in the cartridge to detect target genes as described herein. In some embodiments, the eluate is used to reconstitute at least some of the PCR reagents, which are present in the cartridge as lyophilized particles.

In some embodiments, RT-PCR is used to amplify and analyze the presence of the target genes. In some embodiments, the reverse transcription uses MMLV RT enzyme and an incubation of 5 to 20 minutes at 40° C. to 50° C. In some embodiments, the PCR uses Taq polymerase with hot start function, such as AptaTaq (Roche). In some embodiments, the initial denaturation is at 90° C. to 100° C. for 20 seconds to 5 minutes; the cycling denaturation temperature is 90° C. to 100° C. for 1 to 10 seconds; the cycling anneal and amplification temperature is 60° C. to 75° C. for 10 to 40 seconds; and up to 50 cycles are performed. In some embodiments a different RT may be used. It may be from another organism or may be a natural or engineered variant of an RT enzyme that may be optimized for different temperature incubations.

The present disclosure is not limited to particular primer and/or probe sequences.

### Exemplary Data Analysis

In some embodiments, a computer-based analysis program is used to translate the raw data generated by the detection assay into data of predictive value for a clinician. The clinician can access the predictive data using any suitable means. Thus, in some embodiments, the present disclosure provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present disclosure contemplates any method capable of receiving, processing, and transmitting the information to and from laboratories conducting the assays, information provides, medical personal, and subjects. For example, in some embodiments of the present disclosure, a sample (e.g., a blood sample) is obtained from a subject and submitted to a profiling (e.g., clinical lab at a medical facility), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample or sputum sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

As described herein, both short (0.1 to less than 8 hours or 2-6 hours) and long (8-24 hours or 8-20 hours) incubation of the blood with the antigens can be used in the methods. The cartridges provided herein can have at least two (2) assay definition files (ADFs) with different signature algorithms for short and for long incubation. The ADF can contain all of the information including the algorithm needed to run the assay on an automated instrument. In some embodiments of the methods and systems provided herein, the ADF can include instructions for performing one or more of the following: initiating an assay-specific sample preparation script on the instrument; initiating an assay-specific load cartridge script on the instrument; initiating an assay-specific reaction script on the instrument; initiating an assay-specific data analysis algorithm on the instrument; or deriving a final call for the assay, based on one or more assay-specific result algorithms or scripts.

The profile data is then prepared in a format suitable for interpretation by a treating clinician. For example, rather than providing raw expression data, the prepared format may represent a diagnosis or risk assessment for the subject, with or without recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

### Exemplary Polynucleotides

In some embodiments, polynucleotides are provided. In some embodiments, synthetic polynucleotides are provided. Synthetic polynucleotides, as used herein, refer to polynucleotides that have been synthesized in vitro either chemically or enzymatically. Chemical synthesis of polynucleotides includes, but is not limited to, synthesis using polynucleotide synthesizers, such as OligoPilot (Cytiva), ABI 3900 DNA Synthesizer (Applied Biosystems), and the like. Enzymatic synthesis includes, but is not limited, to producing polynucleotides by enzymatic amplification, e.g., PCR. A polynucleotide may comprise one or more nucleotide analogs (i.e., modified nucleotides) discussed herein.

In various embodiments, a polynucleotide comprises fewer than 500, fewer than 300, fewer than 200, fewer than 150, fewer than 100, fewer than 75, fewer than 50, fewer than 40, or fewer than 30 nucleotides. In various embodiments, a polynucleotide is between 6 and 200, between 8 and 200, between 8 and 150, between 8 and 100, between 8 and 75, between 8 and 50, between 8 and 40, between 8 and 30, between 15 and 100, between 15 and 75, between 15 and 50, between 15 and 40, or between 15 and 30 nucleotides long.

In some embodiments, the polynucleotide is a primer. In some embodiments, the primer is labeled with a detectable moiety. In some embodiments, a primer is not labeled. A primer, as used herein, is a polynucleotide that is capable of selectively hybridizing to a target RNA or to a cDNA reverse transcribed from the target RNA or to an amplicon that has been amplified from a target RNA or a cDNA (collectively referred to as “template”), and, in the presence of the template, a polymerase and suitable buffers and reagents, can be extended to form a primer extension product.

In some embodiments, the polynucleotide is a probe. In some embodiments, the probe is labeled with a detectable moiety. A detectable moiety, as used herein, includes both directly detectable moieties, such as fluorescent dyes, and indirectly detectable moieties, such as members of binding pairs. When the detectable moiety is a member of a binding pair, in some embodiments, the probe can be detectable by incubating the probe with a detectable label bound to the second member of the binding pair. In some embodiments, a probe is not labeled, such as when a probe is a capture probe, e.g., on a microarray or bead. In some embodiments, a probe is not extendable, e.g., by a polymerase. In other embodiments, a probe is extendable.

In some embodiments, the polynucleotide is a FRET probe that in some embodiments is labeled at the 5′-end with a fluorescent dye (donor) and at the 3′-end with a quencher (acceptor), a chemical group that absorbs (i.e., suppresses) fluorescence emission from the dye when the groups are in close proximity (i.e., attached to the same probe). Thus, in some embodiments, the emission spectrum of the dye should overlap considerably with the absorption spectrum of the quencher. In other embodiments, the dye and quencher are not at the ends of the FRET probe.

### Exemplary Polynucleotide Modifications

In some embodiments, the methods of detecting at least one target gene described herein employ one or more polynucleotides that have been modified, such as polynucleotides comprising one or more affinity-enhancing nucleotide analogs. Modified polynucleotides useful in the methods described herein include primers for reverse transcription, PCR amplification primers, and probes. In some embodiments, the incorporation of affinity-enhancing nucleotides increases the binding affinity and specificity of a polynucleotide for its target nucleic acid as compared to polynucleotides that contain only deoxyribonucleotides, and allows for the use of shorter polynucleotides or for shorter regions of complementarity between the polynucleotide and the target nucleic acid.

In some embodiments, affinity-enhancing nucleotide analogs include nucleotides comprising one or more base modifications, sugar modifications and/or backbone modifications.

In some embodiments, modified bases for use in affinity-enhancing nucleotide analogs include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine.

In some embodiments, affinity-enhancing nucleotide analogs include nucleotides having modified sugars such as 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing nucleotide analogs include backbone modifications such as the use of peptide nucleic acids (PNA; e.g., an oligomer including nucleobases linked together by an amino acid backbone). Other backbone modifications include phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, a polynucleotide includes at least one affinity-enhancing nucleotide analog that has a modified base, at least nucleotide (which may be the same nucleotide) that has a modified sugar, and/or at least one internucleotide linkage that is non-naturally occurring.

In some embodiments, an affinity-enhancing nucleotide analog contains a locked nucleic acid (“LNA”) sugar, which is a bicyclic sugar. In some embodiments, a polynucleotide for use in the methods described herein comprises one or more nucleotides having an LNA sugar. In some embodiments, a polynucleotide contains one or more regions consisting of nucleotides with LNA sugars. In other embodiments, a polynucleotide contains nucleotides with LNA sugars interspersed with deoxyribonucleotides. See, e.g., Frieden, M. et al. (2008) Curr. Pharm. Des. 14(11):1138-1142.

### Exemplary Primers

In some embodiments, a primer and primer pairs are used. In some embodiments, a primer is at least 85%, at least 90%, at least 95%, or 100% identical to, or at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the biomarker targets.

In some embodiments, a primer may also comprise portions or regions that are not identical or complementary to the target gene. In some embodiments, a region of a primer that is at least 85%, at least 90%, at least 95%, or 100% identical or complementary to a target gene is contiguous, such that any region of a primer that is not identical or complementary to the target gene does not disrupt the identical or complementary region.

In some embodiments, a primer comprises a portion that is at least 85%, at least 90%, at least 95%, or 100% identical to a region of a target gene. In some such embodiments, a primer that comprises a region that is at least 85%, at least 90%, at least 95%, or 100% identical to a region of the target gene is capable of selectively hybridizing to a cDNA that has been reverse transcribed from the RNA, or to an amplicon that has been produced by amplification of the target gene. In some embodiments, the primer is complementary to a sufficient portion of the cDNA or amplicon such that it selectively hybridizes to the cDNA or amplicon under the conditions of the particular assay being used.

As used herein, “selectively hybridize” means that a polynucleotide, such as a primer or probe, will hybridize to a particular nucleic acid in a sample with at least 5-fold greater affinity than it will hybridize to another nucleic acid present in the same sample that has a different nucleotide sequence in the hybridizing region. Exemplary hybridization conditions are discussed herein, for example, in the context of a reverse transcription reaction or a PCR amplification reaction. In some embodiments, a polynucleotide will hybridize to a particular nucleic acid in a sample with at least 10-fold greater affinity than it will hybridize to another nucleic acid present in the same sample that has a different nucleotide sequence in the hybridizing region.

In some embodiments, a primer is used to reverse transcribe a target RNA, for example, as discussed herein. In some embodiments, a primer is used to amplify a target RNA or a cDNA reverse transcribed therefrom. Such amplification, in some embodiments, is quantitative PCR, for example, as discussed herein.

In some embodiments, a primer comprises a detectable moiety.

In some embodiments, primer pairs are used. Such primer pairs are designed to amplify a portion of a biomarker gene, or an endogenous control such as a sample adequacy control (SAC), or an exogenous control such as a sample processing control (SPC). In some embodiments, a primer pair is designed to produce an amplicon that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 100 to 300 nucleotides long, 100 to 200 nucleotides long, or 100 to 150 nucleotides long.

Design of primers and probes for amplification of RNA fragments may be performed using DNA Software, Inc.'s Visual OMP (Oligonucleotide Modeling Platform). Visual OMP models, in silico, the folding and hybridization of single-stranded nucleic acids by incorporating all public domain thermodynamic parameters as well as proprietary nearest-neighbor and multi-state thermodynamic parameters for DNA, RNA, PNA, and Inosine. This enables the effective design of primers and probes for complex assays such as microarrays, microfluidics applications and multiplex PCR. In silico experiments simulate secondary structures for targets (optimal and suboptimal), primers (optimal and suboptimal), homodimers, and target and primer heterodimers, given specified conditions. Values for melting temperature (Tm), free energy (ΔG), percent bound, and concentrations for all species are calculated. Additionally, Visual OMP predicts the binding efficiency between primers and probes with target(s) in a single or multiplex reaction.

Using this software tool, predicted interactions between oligonucleotides and the different targets may be evaluated thermodynamically and unwanted interactions minimized.

### Exemplary Probes

In various embodiments, methods of measuring the levels of the biomarkers comprise hybridizing nucleic acids of a sample with a probe.

In some embodiments, the probe comprises a portion that is complementary to a target gene, or an endogenous control such as a sample adequacy control (SAC), or an exogenous control such as a sample processing control (SPC). In some embodiments, the probe comprises a portion that is at least 85%, at least 90%, at least 95%, or 100% identical to a region of the target gene.

In some such embodiments, a probe that is at least 85%, at least 90%, at least 95%, or 100% complementary to a target gene is complementary to a sufficient portion of the target gene such that it selectively hybridizes to the target gene under the conditions of the particular assay being used. In some embodiments, a probe that is complementary to a target gene comprises a region that is at least 85%, at least 90%, at least 95%, or 100% complementary to at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the target gene.

A probe that is at least 85%, at least 90%, at least 95%, or 100% complementary to a target gene may also comprise portions or regions that are not complementary to the target gene. In some embodiments, a region of a probe that is at least 85%, at least 90%, at least 95%, or 100% complementary to a target gene is contiguous, such that any region of a probe that is not complementary to the target gene does not disrupt the complementary region.

In some embodiments, the probe comprises a portion that is at least 85%, at least 90%, at least 95%, or 100% identical to a region of the target gene, or an endogenous control such as a sample adequacy control (SAC), or an exogenous control such as a sample processing control (SPC). In some such embodiments, a probe that comprises a region that is at least 85%, at least 90%, at least 95%, or 100% identical to a region of the target gene is capable of selectively hybridizing to a cDNA that has been reverse-transcribed from a target gene or to an amplicon that has been produced by amplification of the target gene. In some embodiments, the probe is at least 85%, at least 90%, at least 95%, or 100% complementary to a sufficient portion of the cDNA or amplicon such that it selectively hybridizes to the cDNA or amplicon under the conditions of the particular assay being used. In some embodiments, a probe that is complementary to a cDNA or amplicon comprises a region that is at least 85%, at least 90%, at least 95%, or 100% complementary to at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the cDNA or amplicon. A probe that is at least 85%, at least 90%, at least 95%, or 100% complementary to a cDNA or amplicon may also comprise portions or regions that are not complementary to the cDNA or amplicon. In some embodiments, a region of a probe that is at least 85%, at least 90%, at least 95%, or 100% complementary to a cDNA or amplicon is contiguous, such that any region of a probe that is not complementary to the cDNA or amplicon does not disrupt the complementary region.

In some embodiments, the method of detecting one or more target genes comprises: (a) reverse transcribing a target RNA to produce a cDNA that is complementary to the target RNA; (b) amplifying the cDNA from (a); and (c) detecting the amount of a target RNA using real time RT-PCR and a detection probe (which may be simultaneous with the amplification step (b)).

As described above, in some embodiments, real time RT-PCR detection may be performed using a FRET probe, which includes, but is not limited to, a TAQMAN® probe, a Molecular beacon probe and a Scorpions probe. In some embodiments, the real time RT-PCR detection is performed with a TAQMAN® probe, i.e., a linear probe that typically has a fluorescent dye covalently bound at one end of the DNA and a quencher molecule covalently bound elsewhere, such as at the other end of, the DNA. The FRET probe comprises a sequence that is complementary to a region of the cDNA or amplicon such that, when the FRET probe is hybridized to the cDNA or amplicon, the dye fluorescence is quenched, and when the probe is digested during amplification of the cDNA or amplicon, the dye is released from the probe and produces a fluorescence signal. In some embodiments, the amount of target gene in the sample is proportional to the amount of fluorescence measured during amplification.

The TAQMAN® probe typically comprises a region of contiguous nucleotides having a sequence that is at least 85%, at least 90%, at least 95%, or 100% identical or complementary to a region of a target gene or its complementary cDNA that is reverse transcribed from the target RNA template (i.e., the sequence of the probe region is complementary to or identically present in the target RNA to be detected) such that the probe is selectively hybridizable to a PCR amplicon of a region of the target gene. In some embodiments, the probe comprises a region of at least 6 contiguous nucleotides having a sequence that is fully complementary to or identically present in a region of a cDNA that has been reverse transcribed from a target gene. In some embodiments, the probe comprises a region that is at least 85%, at least 90%, at least 95%, or 100% identical or complementary to at least 8 contiguous nucleotides, at least 10 contiguous nucleotides, at least 12 contiguous nucleotides, at least 14 contiguous nucleotides, or at least 16 contiguous nucleotides having a sequence that is complementary to or identically present in a region of a cDNA reverse transcribed from a target gene to be detected.

In some embodiments, the region of the amplicon that has a sequence that is at least 85%, at least 90%, at least 95%, or 100% complementary to the TAQMAN® probe sequence is at or near the center of the amplicon molecule. In some embodiments, there are independently at least 2 nucleotides, such as at least 3 nucleotides, such as at least 4 nucleotides, such as at least 5 nucleotides of the amplicon at the 5′-end and at the 3′-end of the region of complementarity.

In some embodiments, Molecular Beacons can be used to detect PCR products. Like TAQMAN® probes, Molecular Beacons use FRET to detect a PCR product via a probe having a fluorescent dye and a quencher attached at the ends of the probe. Unlike TAQMAN® probes, Molecular Beacons remain intact during the PCR cycles. Molecular Beacon probes form a stem-loop structure when free in solution, thereby allowing the dye and quencher to be in close enough proximity to cause fluorescence quenching. When the Molecular Beacon hybridizes to a target, the stem-loop structure is abolished so that the dye and the quencher become separated in space and the dye fluoresces. Molecular Beacons are available, e.g., from Gene Link™ (see genelink.com).

In some embodiments, Scorpion probes can be used as both sequence-specific primers and for PCR product detection. Like Molecular Beacons, Scorpions probes form a stem-loop structure when not hybridized to a target nucleic acid. However, unlike Molecular Beacons, a Scorpions probe achieves both sequence-specific priming and PCR product detection. A fluorescent dye molecule is attached to the 5′-end of the Scorpions probe, and a quencher is attached elsewhere, such as to the 3′-end. The 3′ portion of the probe is complementary to the extension product of the PCR primer, and this complementary portion is linked to the 5′-end of the probe by a non-amplifiable moiety. After the Scorpions primer is extended, the target-specific sequence of the probe binds to its complement within the extended amplicon, thus opening up the stem-loop structure and allowing the dye on the 5′-end to fluoresce and generate a signal. Scorpions probes are available from, e.g., Premier Biosoft International (see premierbiosoft.com).

In some embodiments, labels that can be used on the FRET probes include colorimetric and fluorescent dyes such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′, 4′,5′,7′-Tetrabromosulfonefluorescein, and TET.

Examples of dye/quencher pairs (i.e., donor/acceptor pairs) include, but are not limited to, fluorescein/tetramethylrhodamine; IAEDANS/fluorescein; EDANS/dabcyl; fluorescein/fluorescein; BODIPY FL/BODIPY FL; fluorescein/QSY 7 or QSY 9 dyes. When the donor and acceptor are the same, FRET may be detected, in some embodiments, by fluorescence depolarization. Certain specific examples of dye/quencher pairs (i.e., donor/acceptor pairs) include, but are not limited to, Alexa Fluor 350/Alexa Fluor488; Alexa Fluor 488/Alexa Fluor 546; Alexa Fluor 488/Alexa Fluor 555; Alexa Fluor 488/Alexa Fluor 568; Alexa Fluor 488/Alexa Fluor 594; Alexa Fluor 488/Alexa Fluor 647; Alexa Fluor 546/Alexa Fluor 568; Alexa Fluor 546/Alexa Fluor 594; Alexa Fluor 546/Alexa Fluor 647; Alexa Fluor 555/Alexa Fluor 594; Alexa Fluor 555/Alexa Fluor 647; Alexa Fluor 568/Alexa Fluor 647; Alexa Fluor 594/Alexa Fluor 647; Alexa Fluor 350/QSY35; Alexa Fluor 350/dabcyl; Alexa Fluor 488/QSY 35; Alexa Fluor 488/dabcyl; Alexa Fluor 488/QSY 7 or QSY 9; Alexa Fluor 555/QSY 7 or QSY9; Alexa Fluor 568/QSY 7 or QSY 9; Alexa Fluor 568/QSY 21; Alexa Fluor 594/QSY 21; and Alexa Fluor 647/QSY 21. In some instances, the same quencher may be used for multiple dyes, for example, a broad spectrum quencher, such as an Iowa Black® quencher (Integrated DNA Technologies, Coralville, Iowa) or a Black Hole Quencher™ (BHQ™; Sigma-Aldrich, St. Louis, Mo.).

In some embodiments, for example, in a multiplex reaction in which two or more moieties (such as amplicons) are detected simultaneously, each probe comprises a detectably different dye such that the dyes may be distinguished when detected simultaneously in the same reaction. One skilled in the art can select a set of detectably different dyes for use in a multiplex reaction.

Specific examples of fluorescently labeled ribonucleotides useful in the preparation of PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Cytiva, such as Cy3-UTP and Cy5-UTP.

Examples of fluorescently labeled deoxyribonucleotides useful in the preparation of PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. Fluorescently labeled nucleotides are commercially available and can be purchased from, e.g., Thermo Fisher.

In some embodiments, dyes and other moieties, such as quenchers, are introduced into polynucleotide used in the methods described herein, such as FRET probes, via modified nucleotides. A “modified nucleotide” refers to a nucleotide that has been chemically modified, but still functions as a nucleotide. In some embodiments, the modified nucleotide has a chemical moiety, such as a dye or quencher, covalently attached, and can be introduced into a polynucleotide, for example, by way of solid phase synthesis of the polynucleotide. In other embodiments, the modified nucleotide includes one or more reactive groups that can react with a dye or quencher before, during, or after incorporation of the modified nucleotide into the nucleic acid. In specific embodiments, the modified nucleotide is an amine-modified nucleotide, i.e., a nucleotide that has been modified to have a reactive amine group. In some embodiments, the modified nucleotide comprises a modified base moiety, such as uridine, adenosine, guanosine, and/or cytosine. In specific embodiments, the amine-modified nucleotide is selected from 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP. In some embodiments, nucleotides with different nucleobase moieties are similarly modified, for example, 5-(3-aminoallyl)-GTP instead of 5-(3-aminoallyl)-UTP. Many amine modified nucleotides are commercially available from, e.g., Applied Biosystems, Sigma, Jena Bioscience and TriLink.

Exemplary detectable moieties also include, but are not limited to, members of binding pairs. In some such embodiments, a first member of a binding pair is linked to a polynucleotide. The second member of the binding pair is linked to a detectable label, such as a fluorescent label. When the polynucleotide linked to the first member of the binding pair is incubated with the second member of the binding pair linked to the detectable label, the first and second members of the binding pair associate and the polynucleotide can be detected. Exemplary binding pairs include, but are not limited to, biotin and streptavidin, antibodies and antigens, etc.

In some embodiments, multiple target genes are detected in a single multiplex reaction. In some such embodiments, each probe that is targeted to a unique amplicon is spectrally distinguishable when released from the probe, in which case each target gene is detected by a unique fluorescence signal. In some embodiments, two or more target genes are detected using the same fluorescent signal, in which case detection of that signal indicates the presence of either of the target genes or both.

One skilled in the art can select a suitable detection method for a selected assay, e.g., a real-time RT-PCR assay. The selected detection method need not be a method described above, and may be any method.

### Exemplary Compositions and Kits

In another aspect, compositions are provided. In some embodiments, compositions are provided for use in the methods described herein.

In some embodiments, compositions are provided that comprise at least one target gene-specific primer. The terms “target gene-specific primer” and “target RNA-specific primer” are used interchangeably and encompass primers that have a region of contiguous nucleotides having a sequence that is (i) at least 85%, at least 90%, at least 95%, or 100% identical to a region of a target gene, or (ii) at least 85%, at least 90%, at least 95%, or 100% complementary to the sequence of a region of contiguous nucleotides found in a target gene. In some embodiments, a composition is provided that comprises at least one pair of target gene-specific primers. The term “pair of target gene-specific primers” encompasses pairs of primers that are suitable for amplifying a defined region of a target gene. A pair of target gene-specific primers typically comprises a first primer that comprises a sequence that is at least 85%, at least 90%, at least 95%, or 100% identical to the sequence of a region of a target gene and a second primer that comprises a sequence that is at least 85%, at least 90%, at least 95%, or 100% complementary to a region of a target gene. A pair of primers is typically suitable for amplifying a region of a target gene that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 100 to 300 nucleotides long, 100 to 200 nucleotides long, or 100 to 150 nucleotides long.

In some embodiments, a composition comprises at least one pair of target gene-specific primers. In some embodiments, a composition additionally comprises a pair of target gene-specific primers for amplifying an endogenous control (such as an SAC) and/or one pair of target gene-specific primers for amplifying an exogenous control (such as an SPC).

In some embodiments, a composition comprises at least one target gene-specific probe. The terms “target gene-specific probe” and “target RNA-specific probe” are used interchangeably and encompass probes that have a region of contiguous nucleotides having a sequence that is (i) at least 85%, at least 90%, at least 95%, or 100% identical to a region of a target gene, or (ii) at least 85%, at least 90%, at least 95%, or 100% complementary to the sequence of a region of contiguous nucleotides found in a target gene.

In some embodiments, a composition (including a composition described above that comprises one or more pairs of target gene-specific primers) comprises one or more probes for detecting the target genes. In some embodiments, a composition comprises a probe for detecting an endogenous control (such as an SAC) and/or a probe for detecting an exogenous control (such as an SPC).

In some embodiments, a composition is an aqueous composition. In some embodiments, the aqueous composition comprises a buffering component, such as phosphate, tris, HEPES, etc., and/or additional components, as discussed below. In some embodiments, a composition is dry, for example, lyophilized, and suitable for reconstitution by addition of fluid. A dry composition may include one or more buffering components and/or additional components.

In some embodiments, a composition further comprises one or more additional components. Additional components include, but are not limited to, salts, such as NaCl, KCl, and MgCl₂; polymerases, including thermostable polymerases such as Taq; dNTPs; reverse transcriptases, such as MMLV reverse transcriptase; Rnase inhibitors; bovine serum albumin (BSA) and the like; reducing agents, such as β-mercaptoethanol; EDTA and the like; etc. One skilled in the art can select suitable composition components depending on the intended use of the composition.

In some embodiments, compositions are provided that comprise at least one polynucleotide for detecting at least one target gene. In some embodiments, the polynucleotide is used as a primer for a reverse transcriptase reaction. In some embodiments, the polynucleotide is used as a primer for amplification. In some embodiments, the polynucleotide is used as a primer for PCR. In some embodiments, the polynucleotide is used as a probe for detecting at least one target gene. In some embodiments, the polynucleotide is detectably labeled. In some embodiments, the polynucleotide is a FRET probe. In some embodiments, the polynucleotide is a TAQMAN® probe, a Molecular Beacon, or a Scorpions probe.

In some embodiments, a composition comprises at least one FRET probe having a sequence that is at least 85%, at least 90%, at least 95%, or 100% identical, or at least 85%, at least 90%, at least 95%, or 100% complementary, to a region of a target gene. In some embodiments, a FRET probe is labeled with a donor/acceptor pair such that when the probe is digested during the PCR reaction, it produces a unique fluorescence emission that is associated with a specific target gene. In some embodiments, when a composition comprises multiple FRET probes, each probe is labeled with a different donor/acceptor pair such that when the probe is digested during the PCR reaction, each one produces a unique fluorescence emission that is associated with a specific probe sequence and/or target gene. In some embodiments, the sequence of the FRET probe is complementary to a target region of a target gene. In other embodiments, the FRET probe has a sequence that comprises one or more base mismatches when compared to the sequence of the best-aligned target region of a target gene.

In some embodiments, a composition comprises a FRET probe consisting of at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides, wherein at least a portion of the sequence is at least 85%, at least 90%, at least 95%, or 100% identical, or at least 85%, at least 90%, at least 95%, or 100% complementary, to a region of, a target gene. In some embodiments, at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides of the FRET probe are identically present in, or complementary to a region of, a target gene. In some embodiments, the FRET probe has a sequence with one, two or three base mismatches when compared to the sequence or complement of the target gene.

In some embodiments, a kit comprises a polynucleotide discussed above. In some embodiments, a kit comprises at least one primer and/or probe discussed above. In some embodiments, a kit comprises at least one polymerase, such as a thermostable polymerase. In some embodiments, a kit comprises dNTPs. In some embodiments, kits for use in the real time RT-PCR methods described herein comprise one or more target gene-specific FRET probes and/or one or more primers for reverse transcription of target RNAs and/or one or more primers for amplification of target genes or cDNAs reverse transcribed therefrom.

In some embodiments, one or more of the primers and/or probes is “linear”. A “linear” primer refers to a polynucleotide that is a single stranded molecule, and typically does not comprise a short region of, for example, at least 3, 4 or 5 contiguous nucleotides, which are complementary to another region within the same polynucleotide such that the primer forms an internal duplex. In some embodiments, the primers for use in reverse transcription comprise a region of at least 4, such as at least 5, such as at least 6, such as at least 7 or more contiguous nucleotides at the 3′-end that has a sequence that is complementary to region of at least 4, such as at least 5, such as at least 6, such as at least 7 or more contiguous nucleotides at the 5′-end of a target gene.

In some embodiments, a kit comprises one or more pairs of linear primers (a “forward primer” and a “reverse primer”) for amplification of a target gene or cDNA reverse transcribed therefrom. Accordingly, in some embodiments, a first primer comprises a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides having a sequence that is at least 85%, at least 90%, at least 95%, or 100% identical to the sequence of a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides at a first location in the target gene. Furthermore, in some embodiments, a second primer comprises a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides having a sequence that is at least 85%, at least 90%, at least 95%, or 100% complementary to the sequence of a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides at a second location in the target gene, such that a PCR reaction using the two primers results in an amplicon extending from the first location of the target gene to the second location of the target gene.

In some embodiments, the kit comprises at least two, at least three, or at least four sets of primers, each of which is for amplification of a different target gene or cDNA reverse transcribed therefrom. In some embodiments, the kit further comprises at least one set of primers for amplifying a control RNA, such as an endogenous control and/or an exogenous control.

In some embodiments, probes and/or primers for use in the compositions described herein comprise deoxyribonucleotides. In some embodiments, probes and/or primers for use in the compositions described herein comprise deoxyribonucleotides and one or more nucleotide analogs, such as LNA analogs or other duplex-stabilizing nucleotide analogs described above. In some embodiments, probes and/or primers for use in the compositions described herein comprise all nucleotide analogs. In some embodiments, the probes and/or primers comprise one or more duplex-stabilizing nucleotide analogs, such as LNA analogs, in the region of complementarity.

In some embodiments, the kits for use in real time RT-PCR methods described herein further comprise reagents for use in the reverse transcription and amplification reactions. In some embodiments, the kits comprise enzymes, such as a reverse transcriptase or a heat stable DNA polymerase, such as Taq polymerase. In some embodiments, the kits further comprise deoxyribonucleotide triphosphates (dNTP) for use in reverse transcription and/or in amplification. In further embodiments, the kits comprise buffers optimized for specific hybridization of the probes and primers.

A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the methods described herein. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some embodiments, the kit can comprise the reagents described above provided in one or more GENEXPERT® cartridge(s). These cartridges permit extraction, amplification, and detection to be carried out within this self-contained “laboratory in a cartridge.” (See e.g., U.S. Pat. Nos. 5,958,349, 6,403,037, 6,440,725, 6,783,736, 6,818,185; each of which is herein incorporated by reference in its entirety.) Reagents for measuring genomic copy number level and detecting a pathogen could be provided in separate cartridges within a kit or these reagents (adapted for multiplex detection) could be provide in a single cartridge.

Any of the kits described here can include, in some embodiments, a receptacle for a blood sample. The receptacle may contain one or more antigens or it may be a Li-heparin tube that does not include antigens.

The following examples are for illustration purposes only, and are not meant to be limiting in any way.

### Examples

### Example 1: MTB Infected Vs. Not Infected Using PCA Analysis

RNA-Seq analysis was performed to identify markers that were differentially expressed in ATB vs. LTBI samples and MTB infected vs. MTB not-infected samples to identify a set of 26 potential markers and 4 reference genes for further testing. All 30 genes were analyzed by RT-PCR analysis to measure expression levels in a set samples of known status (MTB infected vs. MTB not-infected and ATB vs. LTBI). Those data were then analyzed using PCA. Good separation in PCA was found for MTB infected vs. MTB not-infected using 4, 6, or 8 of the markers as follows: 4-marker: IFN-γ, IP10, MIG and IL2, 6-marker: IFN-γ, MIG, IL2, SERPING1, LINC01093, and GBP1P1; and 8-marker: IFN-γ, MIG, IL2, SERPING1, LINC01093, GBP1P1, VEGFA and GBP5. For ATB vs. LTBI two 4 marker sets were identified: signature 1: VEGFA, LINC01093, SERPING1 and GB5; and signature 2: VEGFA, LINC01093, SERPING1, and GBP1P1.

### Example 2: MTB Infected Vs. Not-Infected Using ROC Analysis

The expression level of the 30 candidate genes was measured in a collection of 35 samples known to be MTB infected and 77 samples know to be MTB not-infected. A down-selection to 15 main candidate markers was performed based on qPCR performance and additional considerations, such as antigenic stimulation kinetics data and biological pathway analysis. For each marker a ΔCt value was calculated for the marker in the sample as compared to a reference gene. For each marker ROC analysis was performed and 9 of the 15 markers showed an AUC greater than 0.9 when analyzing the ΔCt between the marker gene and a reference gene. The AUCs are shown in Table 1.

TABLE 1
MTB infected vs. Not-infected

| | | Standard | Lower bound | Upper bound |
|---|---|---|---|---|
| Marker | AUC | error | (95%) | (95%) |
| ANKRD22 | 0.968 | 0.013 | 0.943 | 0.994 |
| GBP1P1 | 0.952 | 0.022 | 0.908 | 0.996 |
| IP10 | 0.948 | 0.026 | 0.898 | 0.999 |
| FOXP3 | 0.940 | 0.026 | 0.889 | 0.990 |
| MIG | 0.935 | 0.033 | 0.869 | 1.000 |
| IL2 | 0.931 | 0.030 | 0.872 | 0.990 |
| SERPING1 | 0.928 | 0.024 | 0.881 | 0.975 |
| IFNγ | 0.926 | 0.036 | 0.856 | 0.996 |
| GBP5 | 0.912 | 0.029 | 0.856 | 0.969 |
| PLAU | 0.803 | 0.048 | 0.708 | 0.898 |
| DUSP3 | 0.732 | 0.049 | 0.635 | 0.829 |
| PTGS2 | 0.729 | 0.053 | 0.626 | 0.832 |
| SLPI | 0.702 | 0.055 | 0.593 | 0.811 |
| IL10 | 0.615 | 0.056 | 0.505 | 0.726 |
| VEGFA | 0.555 | 0.057 | 0.444 | 0.666 |

Different marker combinations were tested using the ROC analysis, in each case normalized to CD3E. The AUCs for the combinations were as follows: IFN-γ with IL2=0.94, IFN-γ, IL2, CISH, and SERPING1=0.96, IFN-γ, IL2, VEGFA, and MIG=0.95, IFN-γ, IL2, GBP5, and DUSP3=0.95, IFN-γ, PLAU, SLPI and MIG=0.93 and IP10, MIG, FoxP3 and FLT1=0.97.

Additional 4 gene combinations were and shown to have high sensitivity and specificity (sensitivity greater than 90% and specificity greater than 97%) along with an AUC greater than 0.9, including: ANKRD22, GBP1P1, IP10 and FOXP3 (AUC=0.963), GBP1P1, FOXP3, MIG and IL2 (AUC=0.950), IFN-g, GBP1P1, MIG and IL2 (AUC=0.942), and MIG, IFN-g, IP10 and IL2 (AUC=0.940).

### Example 3: ATB Vs. LTBI Using ROC Analysis

The expression level of the 30 candidate genes was measured in a collection of 14 samples known to have ATB and 21 samples known to have LTBI. A down-selection to 15 main candidate markers was performed based on qPCR performance and additional considerations, such as antigenic stimulation kinetics data and biological pathway analysis. For each marker a ΔCt value was calculated for the marker in the sample as compared to a reference gene. For each marker ROC analysis was performed and 8 of the 15 markers showed an AUC greater than 0.7 when analyzing the ΔCt between the marker gene and a reference gene. The AUCs are shown in Table 2.

TABLE 2
ATB vs. LTBI

| | | Standard | Lower | Upper |
|---|---|---|---|---|
| Marker | AUC | error | bound (95%) | bound (95%) |
| SLPI | 0.833 | 0.073 | 0.690 | 0.977 |
| VEGFA | 0.806 | 0.074 | 0.661 | 0.951 |
| PLAU | 0.806 | 0.075 | 0.659 | 0.953 |
| GBP5 | 0.793 | 0.086 | 0.624 | 0.961 |
| DUSP3 | 0.793 | 0.090 | 0.616 | 0.969 |
| SERPING1 | 0.769 | 0.090 | 0.591 | 0.946 |
| GBP1P1 | 0.752 | 0.087 | 0.581 | 0.922 |
| PTGS2 | 0.697 | 0.089 | 0.524 | 0.871 |
| ANKRD22 | 0.680 | 0.103 | 0.478 | 0.883 |
| IL2 | 0.612 | 0.096 | 0.424 | 0.801 |
| FOXP3 | 0.585 | 0.100 | 0.390 | 0.780 |
| IL10 | 0.582 | 0.101 | 0.384 | 0.780 |
| IFNg | 0.541 | 0.100 | 0.344 | 0.738 |
| MIG | 0.517 | 0.106 | 0.309 | 0.725 |
| IP10 | 0.510 | 0.105 | 0.304 | 0.717 |

Different marker combinations were tested using the ROC analysis, in each case normalized to CD3E. The AUCs for the combinations were as follows: PLAU, SLPI, VEGFA and GBP5=0.84, PLAU, VEGFA and GBP5=0.83, and PLAU, VEGFA, LINC01093 and SERPING1=0.75.

Additional 4 gene combinations were and shown to have good sensitivity and specificity (sensitivity greater than 64% and specificity greater than 95%) along with an AUC greater than 0.8, including: SLPI, VEGFA, PLAU and GBP5 (AUC=0.854), VEGFA, PLAU, DUSP3 and SERPING 1 (AUC=0.840), SLPI, DUSP3, PLAU and GBP1P1 (AUC=0.830), VEGFA, PLAU, IL2 and SLPI (AUC=0.816) and VEGFA, PLAU, GBP1P1 and SERPING 1 (AUC=0.810),

### Example 4: MTB Infected Vs. Not-Infected Using ROC Analysis

The expression level of the 15 candidate genes was measured in a collection of 336 samples known to be MTB infected or 375 samples know to be MTB not-infected. For each marker a ΔCt value was calculated for the marker in the sample as compared to a reference gene. For each marker ROC analysis was performed and 9 of the 15 markers showed an AUC greater than 0.8 when analyzing the ΔCt between the marker gene and a reference gene. The 4 markers with the highest AUC results, MIG, IFN-γ, IP10, and IL2, were used in a combined analysis to obtain an AUC of 0.939 for the average ΔCt, providing an improved AUC over the highest single gene AUC of 0.915 observed for MIG alone. In one aspect the method for differentiating between ATB and LTBI or LTBI and no TB is characterized by an area under the receiver operator characteristic (ROC) curve (AUC) ranging from 0.7 to 1.

### Example 5: ATB Vs. LTBI Using ROC Analysis

The expression level of the 15 candidate genes was measured in a collection of 162 ATB infected samples known to be MTB infected and 174 LTBI samples. For each marker a ΔCt value was calculated for the marker in the sample as compared to a reference gene. For each marker ROC analysis was performed and 6 of the 15 markers showed an AUC greater than 0.7 when analyzing the ΔCt between the marker gene and a reference gene. The 4 markers with the highest AUC results, VEGFA, PLAU, IL2 and SLPI, were used in a combined analysis to obtain an AUC of 0.823, providing an improved AUC over the highest single gene AUC of 0.792.

### Example 6: Support Vector Machines

Support vector machine analysis was used to evaluate variable importance of the 15 single gene markers in comparison to the ROC results using the ΔCt results from Examples 4 and 5. The top 6 markers identified were the same for both analyses. MIG, IL2, IFN-γ, IP10, GBP1P1 and ANKRD22 were the top 6 for MTB Infected vs. not-infected and VEGFA, PLAU, DUSP3, IL2, SLPI and GBP5 were the top 6 for ATB vs. LTBI.

### Example 7: Random Forest Modelling

Random Forest Modelling was used to evaluate variable importance of the 15 single gene markers from Examples 2 and 3. The model ranks each market by importance and accuracy vs. test data. For the MTB infected vs. not-infected samples the top 4 markers were the ANKRD22, IP10, MIG, IL2 for Random Forest Modelling compared to ANKRD22, GBP1P1, IP10, and FoxP3 for ROC analysis. For the ATB vs. LTBI, 2 of the top 4 marker genes, IL2 and PLAU also showed high accuracy, with IL2 and FoxP3 replacing VEGFA and GBP5 in the top 4 in this analysis. The R-CARET package was used. (Classification And REgression Training=CARET) This package contains functions to streamline the model training process for complex regression and classification problems. The results are shown in Table

TABLE 3

| MTB inf vs. Not-inf | | ATB vs. LTBI | |
|---|---|---|---|
| Marker | Importance | Marker | Importance |
| ANKRD22 | 9.35 | SLPI | 8.91 |
| IP10 | 9.09 | PLAU | 3.99 |
| MIG | 8.89 | IL2 | 3.09 |
| IL2 | 8.61 | FOXP3 | 3.06 |
| IFNg | 6.12 | SERPING2 | 2.12 |
| FOXP3 | 5.92 | VEGFA | 1.81 |
| GBP1P1 | 5.79 | GBP1P1 | 1.49 |
| GBP5 | 5.24 | IL10 | 1.34 |
| PLAU | 5.08 | DUSP3 | 1.21 |
| SERPING1 | 4.12 | GBP5 | 0.98 |
| SLPI | 2.52 | ANKRD22 | 0.54 |
| PTGS2 | 1.36 | PTGS2 | −0.50 |
| VEGFA | −0.23 | IP10 | −0.91 |
| DUSP3 | −0.55 | MIG | −1.36 |
| IL10 | −1.15 | IFNg | −1.36 |

### Example 8. Random Forest Modelling

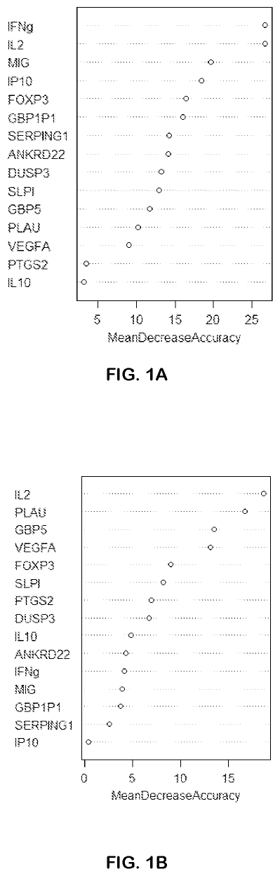

Random Forest Modelling was used to evaluate variable importance of the 15 single gene markers from Examples 4 and 5. For the MTB infected vs. not-infected samples, IFN-γ, IL2, MIG and IP10 were the top 4 markers for both analyses. For the ATB vs. LTBI samples, IL2, PLAU and VEGFA were among the top 4, with GBP5 replacing SLPI in the Random Forest Modelling. The data are illustrated in FIGS. 1A and 1B. FIG. 1A shows the results for MTB infected vs. not-infected and FIG. 1B shows the results for ATB vs. LTBI.

### Example 9. Prototype GeneXpert Cartridge Analysis

To demonstrate proof-of-principle for translation of mRNA signatures for antigen-stimulated blood into a Cepheid GeneXpert compatible assay, we developed two 6-color prototype cartridges. Each prototype cartridge contains four candidate markers, a reference gene for normalization (ΔCt) and a Sample Processing Control (SPC) to control for efficient nucleic acid recovery and detection of possible PCR inhibition. These cartridges can be used to analyze both fresh, antigen-stimulated blood, and antigen-stimulated blood stabilized with for example PAXgene buffer or with a Cepheid lysis buffer before being frozen. PAXgene buffer-stabilized and frozen blood were used for the samples in this example. These cartridges contain all necessary reagents for lysis of the blood sample, isolation of nucleic acid, and qRT-PCR with real-time detection.

The 8 target biomarkers were measured by RT-PCR in samples of known diagnosis and then ROC analysis was performed to determine individual AUCs for each marker tested. The samples were tested using both the LIOFeron TB/LTBI (Lionex) antigen stimulation or QuantiFERON-TB (Qiagen) with either 3-4 or 16-20 hours of stimulation. The results are shown in Tables 4 and 5.

TABLE 4
MTB infected vs. Not-infected

| 3-4 hour incubation | | | | 16-20 hour incubation | | | |
|---|---|---|---|---|---|---|---|
| Lionex | | QFT-TB1 | | Lionex | | QFT-TB1 | |
| Marker | AUC | Marker | AUC | Marker | AUC | Marker | AUC |
| MIG | 0.857 | IL2 | 0.937 | MIG | 0.937 | MIG | 0.986 |
| IL2 | 0.829 | MIG | 0.890 | IL2 | 0.921 | IL2 | 0.952 |
| IFNg | 0.794 | IFNg | 0.843 | SLPI | 0.870 | IFNg | 0.886 |
| GBP5 | 0.645 | GBP5 | 0.676 | GBP5 | 0.824 | GBP5 | 0.875 |
| VEGFA | 0.605 | SERPING1 | 0.613 | IFNg | 0.814 | SERPING1 | 0.861 |
| SERPING1 | 0.599 | SLPI | 0.568 | PLAU | 0.798 | SLPI | 0.773 |
| SLPI | 0.559 | VEGFA | 0.537 | SERPING1 | 0.782 | PLAU | 0.638 |
| PLAU | 0.514 | PLAU | 0.513 | VEGFA | 0.594 | VEGFA | 0.602 |

TABLE 5
ATB vs. LTBI

| 3-4 hour incubation | | | | 16-20 hour incubation | | | |
|---|---|---|---|---|---|---|---|
| Lionex | | QFT-TB1 | | Lionex | | QFT-TB1 | |
| Marker | AUC | Marker | AUC | Marker | AUC | Marker | AUC |
| MIG | 0.965 | GBP5 | 0.969 | SERPING1 | 0.950 | PLAU | 0.915 |
| GBP5 | 0.962 | MIG | 0.946 | SLPI | 0.896 | SERPING1 | 0.896 |
| SERPING1 | 0.938 | SERPING1 | 0.938 | GBP5 | 0.869 | MIG | 0.892 |
| PLAU | 0.923 | IFNg | 0.904 | MIG | 0.858 | GBP5 | 0.835 |
| IFNg | 0.915 | IL2 | 0.877 | VEGFA | 0.858 | SLPI | 0.827 |
| IL2 | 0.912 | PLAU | 0.815 | PLAU | 0.854 | VEGFA | 0.815 |
| VEGFA | 0.869 | SLPI | 0.815 | IFNg | 0.635 | IFNg | 0.712 |
| SLPI | 0.808 | SLPI | 0.808 | IL2 | 0.619 | IL2 | 0.523 |

### Example 10. Prototype GeneXpert Cartridge Analysis

A 10-color prototype cartridge was developed to demonstrate proof-of-principle for translation of mRNA signatures into a Cepheid GeneXpert compatible assay. The prototype cartridge contained nine candidate markers, a reference gene for normalization (ΔCt) and a Sample Processing Control (SPC) to control for efficient nucleic acid recovery and detection of possible PCR inhibition. These cartridges can be used to analyze both fresh, antigen-stimulated blood, and antigen-stimulated blood stabilized with, for example, PAXgene buffer or with a lysis buffer before being frozen. PAXgene buffer-stabilized and frozen blood were used for the samples in this example. These cartridges contain all necessary reagents for lysis of the blood sample, isolation of nucleic acid, and qRT-PCR with real-time detection.

The 9 target biomarkers were measured by RT-PCR in samples of known diagnosis and then ROC analysis was performed to determine individual AUCs for each marker tested. The samples were tested using both the LIOFeron TB/LTBI (Lionex) antigen stimulation or QuantiFERON-TB (Qiagen) with either 3-4 or 16-20 hours of stimulation. The results are shown in Tables 6 and 7.

TABLE 6
ROC Analysis-MTB Infected vs. Not Infected
MTB infected vs. Not-infected

| Singleplex qPCR | | | 10-Color Cartridge Multiplex | | |
|---|---|---|---|---|---|
| | | Std. | | | Std. |
| Marker | AUC | Error | Marker | AUC | Error |
| IL2 | 0.885 | 0.017 | IL2 | 0.902 | 0.015 |
| IFNg | 0.871 | 0.018 | MIG | 0.887 | 0.016 |
| MIG | 0.861 | 0.019 | IFNg | 0.886 | 0.017 |
| GBP1P1 | 0.801 | 0.023 | GBP1P1 | 0.761 | 0.027 |
| GBP5 | 0.746 | 0.027 | SLPI | 0.748 | 0.025 |
| SLPI | 0.681 | 0.027 | GBP5 + DUSP3 | 0.738 | 0.026 |
| PLAU | 0.672 | 0.027 | PLAU | 0.663 | 0.028 |
| DUSP3 | 0.624 | 0.031 | VEGFA | 0.538 | 0.030 |
| VEGFA | 0.549 | 0.030 | | | |
| Core Set (IL2, IFNg, | 0.891 | 0.017 | Core Set (IL2, IFNg, | 0.892 | 0.017 |
| MIG, and GBP1P1) | | | MIG, and GBP1P1) | | |

TABLE 7
ROC Analysis-ATB vs. LTBI
ATB vs. LTBI

| Singleplex qPCR | | | 10-Color Cartridge Multiplex | | |
|---|---|---|---|---|---|
| | | Std. | | | Std. |
| Marker | AUC | Error | Marker | AUC | Error |
| SLPI | 0.798 | 0.032 | GBP5 + DUSP3 | 0.759 | 0.035 |
| DUSP3 | 0.769 | 0.038 | SLPI | 0.726 | 0.038 |
| GBP5 | 0.726 | 0.037 | PLAU | 0.709 | 0.039 |
| PLAU | 0.704 | 0.039 | GBP1P1 | 0.768 | 0.039 |
| GBP1P1 | 0.698 | 0.039 | MIG | 0.661 | 0.041 |
| MIG | 0.659 | 0.041 | VEGFA | 0.605 | 0.042 |
| IFNg | 0.641 | 0.042 | IFNg | 0.598 | 0.043 |
| VEGFA | 0.600 | 0.042 | IL2 | 0.574 | 0.043 |
| IL2 | 0.517 | 0.044 | | | |
| PLAU, SLPI, DUSP3, | 0.797 | 0.036 | PLAU, SLPI, DUSP3, | 0.784 | 0.034 |
| & GBP5 | | | & GBP5 | | |
| PLAU, SLPI, | 0.747 | 0.039 | PLAU, SLPI, | 0.737 | 0.037 |
| DUSP3/GBP5, | | | DUSP3/GBP5, | | |
| VEGFA, & IL2 | | | VEGFA, & IL2 | | |
| All markers | 0.719 | 0.041 | All markers | 0.718 | 0.034 |

The 10-color multiplex cartridge classifies the samples with the same performance as the singleplex PCR, for both signatures. Both peptides (QFT-Plus-TB1 tube from Qiagen) and full-length recombinant proteins (CEPHEID-IGRA tube from Lionex) can be used in the assays. Both short (3-4 h) and long (16-24 h) incubation can be used. There is a slight tendency that the MTB Infected vs. The not Infected signature performed slightly better at long incubation, while some ATB vs. LTBI signature markers performed better at the short incubation time. It is feasible to have 1 cartridge with 2 ADFs with different signature algorithms (for e.g., markers and/or coefficients/weighting) for short and for long incubation.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that changes can be made without departing from the spirit and scope of the invention(s).

What is claimed is:

1. A method for (i) diagnosing a patient as being infected with tuberculosis and (ii) determining if the infected patient has ATB or LTBI in a single assay, the method comprising:
(a) obtaining a biological sample from the patient;
(b) exposing the sample to MTB antigens for at least 0.1 hour in a single tube to obtain an antigen stimulated sample;
(c) measuring levels of expression of at least 3 biomarkers selected from IFN-γ, MIG, IP10, IL2, FoxP3, PLAU, SLPI, VEGFA, DUSP3, GBP5, GBP1P1, ANKRD22, SERPING1, PTGS2, and IL10 in the biological sample;
(d) comparing the levels of expression of each of the at least 3 biomarkers to a control,
(e) diagnosing the patient as being infected with tuberculosis based on the expression level of a first combination of 2 or more of the at least 3 biomarkers, and
(f) determining if the patient has ATB or LTBI based on the expression levels of a second combination of 2 or more of the at least 3 biomarker.

2. The method of claim 1, wherein the biological sample comprises whole blood or PBMCs.

3. The method of claim 1, wherein the at least one antigen is selected from CFP-10, ESAT-6, Rv3615, TB7.7, Ala-DH, or epitopes thereof.

4. The method of claim 1, wherein the expression of VEGFA, PLAU, DUSP3, GBP5, GBP1P1, IL2, MIG, SLPI, and IFN-γ are measured and the first combination comprises GBP1P1, MIG, IL2 and IFN-γ and the second combination comprises VEGFA, GBP1P1, GBP5, DUSP3, PLAU, and SLPI.

5. The method of claim 1, wherein the expression of VEGFA, PLAU, DUSP3, IL2, MIG, and IFN-γ are measured and the first combination comprises MIG, IL2 and IFN-γ and the second combination comprises VEGFA, PLAU and DUSP3.

6. The method of claim 1, further comprising evaluating disease severity in a patient that has ATB by comparing the level of expression of the biomarkers in the second combination of biomarkers to a reference value, wherein increased levels of expression are correlated with increased disease severity.

7. The method of claim 1, further comprising treating the patient by
(e) administering an effective amount of at least one antibiotic to the patient.

8. A method for (i) diagnosing a patient as being infected with MTB or not-infected in a first analysis and (ii) determining if the patient has ATB or LTBI in a second analysis, the method comprising:
(a) obtaining a biological sample from the patient;
(b) exposing the biological sample to MTB antigens for at least 0.1 hour in a single tube to obtain an antigen stimulated sample;
(c) measuring levels of expression of at least 3 biomarkers selected from IFN-γ, MIG, IP10, IL2, FoxP3, PLAU, SLPI, VEGFA, DUSP3, GBP5, GBP1P1, ANKRD22, SERPING1, PTGS2, and IL10 in the antigen stimulated sample;
(d) performing a first statistical analysis of a first set of the biomarkers measured in step (c);
(e) performing a second statistical analysis of a second set of the biomarkers measured in step (c);
(f) diagnosing the patient as being infected with MTB, not infected with MTB or having an inconclusive diagnosis based on the first statistical analysis; and
(g) diagnosing the patient as having ATB or LTBI based on the second statistical analysis.

9. The method of claim 8, wherein the first set of biomarkers is selected from the following sets of biomarkers:
a. IFN-γ, MIG, IL2, GBP1P1;
b. ANKRD22, GBP1P1, IP10, FOXP3;
c. MIG, IL2, GBP1P1, FOXP3;
d. IFN-γ, MIG, IL2, DUSP3;
e. IFN-γ, MIG, IL2, FOXP3, GBP1P1;
f IFN-γ, MIG, IL2, FOXP3;
g. IFN-γ, MIG, IL2, IP10;
h. IFN-γ, MIG, IL2, GBP5;
i. IFN-γ, MIG, IL2, GBP5 with PLAU or SLPI; and
j. IFN-γ, MIG, IL2, PTGS2;
and the second set of biomarkers is selected from the following sets of biomarkers:
i. SLPI, VEGFA, PLAU, GBP5;
ii. SLPI, IL2, PLAU, GBP5;
iii. VEGFA, PLAU, DUSP3, SERPING1;
iv. SLPI, PLAU, DUSP3, GBP1P1;
v. VEGFA, PLAU, IL2, SLPI;
vi. SERPING1, PLAU, VEGFA, GBP1P1;
vii. SLPI, PLAU, GBP5, DUSP3; and
viii. GBP5, SLPI, PLAU, DUSP3, GBP1P1.

10. The method of claim 8, wherein the patient is diagnosed as having an inconclusive diagnosis in step (f) and diagnosed as having ATB in step (g) and wherein the confidence level in the step (g) diagnosis is high because the correlation with the ATB reference is high.

11. The method of claim 8, wherein step (c) comprises measuring levels of expression of IFN-γ, MIG, IL2, PLAU, SLPI, DUSP3, GBP5, and GBP1P1 and optionally VEGFA, in the antigen stimulated sample.

12. The method of claim 8, further comprising treating the patient by
(h) administering an effective amount of at least one antibiotic to the patient.

13. The method of claim 12, wherein if the patient is diagnosed as having ATB, the method further comprises administering an effective amount of a corticosteroid to the patient.

14. The method of claim 12, wherein said at least one antibiotic is selected from the group consisting of rifampicin, isoniazid, pyrazinamide, and ethambutol.

15. The method of claim 8, wherein the biological sample comprises whole blood or PBMCs and wherein the biological sample is stimulated by incubation with at least one tuberculosis antigen before measuring levels of expression of the biomarkers.

16. A method of monitoring a tuberculosis infection in a patient diagnosed according to claim 1, the method comprising:
(a) measuring levels of expression of two or more biomarkers selected from IFN-γ, MIG, IP10, IL2, FoxP3, PLAU, SLPI, VEGFA, DUSP3, GBP5, GBP1P1, ANKRD22, SERPING1, PTGS2, and IL10 biomarkers in a first antigen stimulated biological sample from the subject, wherein the first antigen stimulated biological sample is obtained from the subject at a first time point;
(b) measuring levels of expression of the same two or more biomarkers in a second antigen stimulated biological sample from the subject, wherein the second antigen stimulated biological sample is obtained from the subject at a second time point that is later than the first time point; and
(c) comparing the levels of expression of the biomarkers in the first antigen stimulated biological sample to the levels of expression of the biomarkers in the second antigen stimulated biological sample,
wherein decreased levels of expression of the two or more biomarkers in the second antigen stimulated biological sample compared to the levels of expression of the two or more biomarkers in the first antigen stimulated biological sample indicate that the tuberculosis infection in the patient is improving and increased levels of expression of the two or more biomarkers in the second antigen stimulated biological sample compared to the levels of expression of the biomarkers in the first biological sample indicate that the tuberculosis infection in the patient is worsening.

17. The method of claim 16, wherein monitoring comprises identifying the patient as having LTBI and providing a prognosis that the patient will progress to ATB.

18. The method of claim 16, wherein the step of measuring levels of expression of the two or more biomarkers is carried out according to at least one of the following: (a) before the onset of active tuberculosis in the subject; (b) while the subject is showing symptoms of active tuberculosis; (c) during or after the use of an anti-tuberculosis agent to treat the active tuberculosis; or (d) during or after the use of a preventive treatment for LTBI.

19. The method of claim 16, further comprising selecting a treatment regimen for the patient based on the patient's condition, and treating the patient by administering an effective amount of at least one antibiotic to the patient.

20. A kit comprising primer pairs for amplifying each of at least 4 biomarkers selected from IFN-γ, MIG, IP10, IL2, FoxP3, PLAU, SLPI, VEGFA, DUSP3, GBP5, GBP1P1, ANKRD22, SERPING1, PTGS2, and IL10 biomarkers, and a primer pair for amplifying a control from a sample.

21. The kit of claim 20, further comprising agents for stimulating a sample of blood or PBMCs from a patient using one or more MTB antigens.

22. The kit of claim 21, wherein the antigens comprise ESAT-6, CFP-10 and Ala-DH or at least one epitope of at least on antigenic peptide selected from ESAT-6, CFP-10, Rv3615 and Ala-DH which may be recombinant or native.
